SEQUENCE LISTING (15 sequences)

SEQ ID NO: 1 - DNA, 5901 bp, Bifidobacteriumu lomgum JCM1217
  CDS (1)..(5901)
atgaaaaagaagaagactatatcggctgcgctggcaacagcgttagccttaacctgcatgggcagcgggggaggtactgcgttcgcagtgcccctgtctgatgctgacttgcagactttggcaagtcagattcagcaaatcaacgatacttctgattctgcaactgcttccgagactccttccgcacaagccgatgcggttgaaggctggactattgattccaacatcgctcagggcggcgaaatcctggagatggcaaacggttggctgcacctcaagtccactgcctctaacggtaatgcggcagcgaaccccagctccagcaacaactggccggcagtagccgtatggggcacagattacgacttctccaaggccggctccttccacgccaccatcaaatccccgcaggaaggctccgccaaccgcttcggcttctacctgggctacaacgacccgggcagcggcctgttcatcggctacgattcgggcggctggttctggcagacctacaccggtggcggtagcggcagctggtacagcggtgctcgtatcgctgctccgagcgccaacgaagagcacgacattcgggtctcctggaccgacgccaaggtcgccacactgaccgtggatggccagaaggcattcgatgtcgattactccgcaatgacgaacctctccaacaagcttgccatcaaggccggctcctggaaggagctgaacgaggtcaccgacgtgtacatcaaggacttcccggaggttgtcgaagccgccaagcacgcggtttccggcaaggttgtggacgctggaggcgctgccattgaaggcgcaacagtgcgtttggataagaccaaggtgaagaccggtgcagatggcaccttctccttcgccgacattgaagaaggcgaacacacgctttcgattgccaaggaaggctatgaggacgtctcccagcaggtgacggtctccggcgcggatcttgccatcgatcccatcaccctgaacaaaaccgttcaggtcgcctccgaaacgctgaagaccaagaagatggaagttcagattaagaagaacttcccctctgtgctgcagtacacgatgaccgacggcaaggtgatgtacggccagtccaaggatgtgcgcaccgtcgaaatcaacggcaccaacatcgaactgggcgatgacgacgtgaccttcaagaaggtttccgacaccgaggccacctacacgctgaaggtcaaggatgaggccaagaagattgacgcggtgatcaccgttcagatcacggtcaaggccaaccagctgcacctcaacgtcaccaagatcaagaacaacctgtccgaaggcattcctgagggcaacggtgtggaggagaacgccatccagacgctgtccttcccgaaccagagtctcgtttccgtgcgttccagccaggaaaatgcccaattcactggtgctcgtatgtcttccaacacgcagaagcctggcgataccaacttcgcagtgaccgaagatactaacgtcaccgatagcgactacacctacggcttcatctccggtgctggcctgagtgccggcctgtggagcaactccgagcacgatggcacctatgtggcggctcctgtgcgcggcggcagccagaacacgcgtgtctacgccaccacccagcagactggtgacgccacctccctgggcctggccagcgctccgtggtactaccaccgcacggtcaccgattccaagggcaagaagtacaccgtggccgaaaccgctctgccgcagatggccgtggccatcgccggcgacgagaacgaagacggtgccgtcaactggcaggatggcgcaatcgcctaccgcgacatcatgaacaacccgtacaagtccgaggaagttcccgaactggtggcatggcgtatcgccatgaacttcggctcccaggcgcagaacccgttcctcaccacgcttgacaacgtcaagaaggtggccttgaacaccgacggcctcggccagtccgtgctgctcaagggctacggcaatgaaggccacgactccggccacccggactacggcgatatcggccagcgtctcggcggcgccgacgacatgaacaccatgatggaagagggctccaagtatggcgctcgcttcggtgtgcacgtcaacgcctccgaaatgtatccggaagccaaggccttcagcgaggacatggtgcgccgcaactctgcaggcggcctgagctacggctggaactggcttgatcagggtgtcggcatcgacggcatctacgatctggcatccggttctcgtgtaagccgtttcgctgacctcagcaaggaagtcggcgacaacatggacttcatctacctcgatgtgtggggcaacctgacttcttccggttcggaagattcttgggaaacccgcaagatgagcaagatgatcaacgacaacggctggcgtatgaccaccgaatggggttccggcaacgagtacgactccaccttccagcactgggcagctgatctgacctacggcggctacacctccaagggcgagaactccgaagtgatgcgcttcctgcgcaaccaccagaaggacagctgggttggcgactacccgcaatacggcggcgctgccaacgccccgctgctcggcggctacaacatgaaggacttcgaaggctggcagggccgcaacgactatgccgcctacatcaagaacctgtacacccatgatgtgtccactaagttcatccagcacttcaaggtgacccgctgggtcaacaacccgctgctgaccgccgacaatggcaatgccgctgccgtgtccgacccgaacacgaacaacggcaacgagcagattaccctgaaggattccaacggcaacgttgtagtagtctcccgtggttccaacgacacctctagcgcagcctaccgccagcgcaccatcaccttcaacggcgtgaaggtcgcatccggtgtggtctccgcaggcgatggcagcgccactggcgacgagtcctacctgctgccgtggatgtgggattccttcaccggcaagctggtcaaggattccgagcagaagctctaccactggaacaccaagggcggcaccaccacctggacgctgccggacagctggaagaacctctccagcgtaaaggtgtaccagctcaccgatcagggcaagaccaacgagcagaccgttgccgtctccggcggcaaggtgacgcttaccgctgatgccgaaaccccgtacgtggtgtacaagggcgaagccaagcagatccaggtcaactggagcgaaggcatgcatgtggtagacgccggcttcaacggcggctccaacaccctcaccgacaactggaccgtcagcggctccggcaaggccgaagttgaaggcgacaacaacgccatgctgcgcctgaccggcaaggtcgatgtctcccagcgtctgaccgatctcaaggctggccagaagtacgcgctgtatgttggcgtcgacaaccgctccaccggcgatgcatccgtcaccgtaaccagcggcggcaaggtgctggccaccaactccaccggcaagtccatcgccaagaactacatcaaggcatacggccacaacacgaacagcaatacggaaaatggctccagctacttccagaacatgtacgtgttcttcaccgcgcctgagaacggcgatgccacggtaaccctgtctcacaagagcaccgacggagcacacacctacttcgacgatgtgcgcatcgtggagaaccagtactccggcatcacctatgagaaggacggcacgctgaagtccctcaccaacggattcgaaaacaacgcccagggcatctggccgttcgtggtctccggttccgaaggcgttgaggacaaccgcatccacctctccgagctgcatgctccgttcacgcgggccggttgggatgtcaagaagatggacgatgtgctcgatggcacttggtctgtgaaggttaacggcctgacccagaagggcacgctggtctaccagacgatcccgcagaacgtgaagttcgaggcgggtgccaagtacaaggtgagcttcgactaccagtccggttccgatgacatctacgccatcgctgtgggccagggtgaatactctgccggcagcgtgaagctgaccaacctgaagaaggctctgggtgagaccggcaaggccgagttcgagctgaccggtggcgtcaacggcgattcctggttcggtatttactcgaccgcaaccgcacctgatctgcagggttccaccggcaatgcacaggacttcggcggatacaaggacttcgtgctcgacaacctgaagatcgagcgcatcgagtcccagacccgcaccaaggccgaagcgcaggacaaggtcaaggaaatccgcggcaagtacgattccaagcgtgctgagctctccgatgccgcatggcagcagtatcaggacaccttggtcaaggctcgcgtgctcatcaacaagaatggcgcaaccgctgaggacttcaccaaggcatacgacattctcgtggccctcgacgagtacatgaagaccgctcccggcaacgagagcagcgacaagtacgacgtggcagctgacggctccgatgagctgggtggctacaccgtggccacgggcagcgaagagcctaccgcaggcctgccgagcgaaggcccggccgatctggcacaggatggcaacgacagcacccactggcacaccagctggagcgagaacgcagtcggcaacggcaccgcatggtatcagttcaacctcaacgaaccgaccaccatcaacggcctgcgctacctgccgcgctccggaggtatgaacgccaacggcaagatcaagggctacaagatcacgctcactctggcggatggcaccaccaaggatgtcgtcaccgatgctgagttctccaccaccaccatgtggcagaaggccagcttcgacgccgtcgagaatgtgaccgccgtacgctcgaccgtcctgtcttccgcaggccagagcgactcccaggccaacaagttcgcatccgctgccgaactgcgtttgaccacggaccgcgaggttgaggaagagactgtcgctccggacaagaccgacctcaacgacaccatcgccaaggctaacggtcttaaggaatccgactacacggctgaaagctggactgctctggtcaaggcccgcgaagctgcacaggccgtggcggataacgataaggccaccgcttacgatgtggctctggcgctgacgaacctcgaatccgctatcgctggtctcgagaagaccggtgaggagcctggcccaggcccggttgaggtgaacaagaccgacctgcagactgcagtgaacaaggcaagcaagctcgagaaggccgattacacgaccaactcgtgggaagctttcgccgaggcactgaaggctgcacagcaggtgctcgacaacaagaacgccacccagcaggatgtggataccgcactgagcgctcttcaggacgccatctccaagctggaagctgccaccgagccgaagccgaatccggaaccgggcgtggtggacaaggctgctctgaacgcgaccatcaacaaggccgccgccatcaacctgggtctctacaccgacgactccgccaacgctctgcgcgccgcgctgaagaaggcccgtgaggtctccgacaacagcaacgccacgcagaagcaggtcgacgcagctcgtgaagccctggagaaggcaattgccgctctggtgaagcgtcccgctgccaagggtgatggcaacgttgtttccaacacgggctccgatgtcgccacgatcgctctggctggactgctgctggcaggtgctggcgccgccatcgcctaccgtcgcaatcgcgagcaactgtga

SEQ ID NO: 2 - protein, 1966 aa, Bifidobacteriumu lomgum JCM1217
MKKKKTISAALATALALTCMGSGGGTAFAVPLSDADLQTLASQIQQINDTSDSATASETPSAQADAVEGWTIDSNIAQGGEILEMANGWLHLKSTASNGNAAANPSSSNNWPAVAVWGTDYDFSKAGSFHATIKSPQEGSANRFGFYLGYNDPGSGLFIGYDSGGWFWQTYTGGGSGSWYSGARIAAPSANEEHDIRVSWTDAKVATLTVDGQKAFDVDYSAMTNLSNKLAIKAGSWKELNEVTDVYIKDFPEVVEAAKHAVSGKVVDAGGAAIEGATVRLDKTKVKTGADGTFSFADIEEGEHTLSIAKEGYEDVSQQVTVSGADLAIDPITLNKTVQVASETLKTKKMEVQIKKNFPSVLQYTMTDGKVMYGQSKDVRTVEINGTNIELGDDDVTFKKVSDTEATYTLKVKDEAKKIDAVITVQITVKANQLHLNVTKIKNNLSEGIPEGNGVEENAIQTLSFPNQSLVSVRSSQENAQFTGARMSSNTQKPGDTNFAVTEDTNVTDSDYTYGFISGAGLSAGLWSNSEHDGTYVAAPVRGGSQNTRVYATTQQTGDATSLGLASAPWYYHRTVTDSKGKKYTVAETALPQMAVAIAGDENEDGAVNWQDGAIAYRDIMNNPYKSEEVPELVAWRIAMNFGSQAQNPFLTTLDNVKKVALNTDGLGQSVLLKGYGNEGHDSGHPDYGDIGQRLGGADDMNTMMEEGSKYGARFGVHVNASEMYPEAKAFSEDMVRRNSAGGLSYGWNWLDQGVGIDGIYDLASGSRVSRFADLSKEVGDNMDFIYLDVWGNLTSSGSEDSWETRKMSKMINDNGWRMTTEWGSGNEYDSTFQHWAADLTYGGYTSKGENSEVMRFLRNHQKDSWVGDYPQYGGAANAPLLGGYNMKDFEGWQGRNDYAAYIKNLYTHDVSTKFIQHFKVTRWVNNPLLTADNGNAAAVSDPNTNNGNEQITLKDSNGNVVVVSRGSNDTSSAAYRQRTITFNGVKVASGVVSAGDGSATGDESYLLPWMWDSFTGKLVKDSEQKLYHWNTKGGTTTWTLPDSWKNLSSVKVYQLTDQGKTNEQTVAVSGGKVTLTADAETPYVVYKGEAKQIQVNWSEGMHVVDAGFNGGSNTLTDNWTVSGSGKAEVEGDNNAMLRLTGKVDVSQRLTDLKAGQKYALYVGVDNRSTGDASVTVTSGGKVLATNSTGKSIAKNYIKAYGHNTNSNTENGSSYFQNMYVFFTAPENGDATVTLSHKSTDGAHTYFDDVRIVENQYSGITYEKDGTLKSLTNGFENNAQGIWPFVVSGSEGVEDNRIHLSELHAPFTRAGWDVKKMDDVLDGTWSVKVNGLTQKGTLVYQTIPQNVKFEAGAKYKVSFDYQSGSDDIYAIAVGQGEYSAGSVKLTNLKKALGETGKAEFELTGGVNGDSWFGIYSTATAPDLQGSTGNAQDFGGYKDFVLDNLKIERIESQTRTKAEAQDKVKEIRGKYDSKRAELSDAAWQQYQDTLVKARVLINKNGATAEDFTKAYDILVALDEYMKTAPGNESSDKYDVAADGSDELGGYTVATGSEEPTAGLPSEGPADLAQDGNDSTHWHTSWSENAVGNGTAWYQFNLNEPTTINGLRYLPRSGGMNANGKIKGYKITLTLADGTTKDVVTDAEFSTTTMWQKASFDAVENVTAVRSTVLSSAGQSDSQANKFASAAELRLTTDREVEEETVAPDKTDLNDTIAKANGLKESDYTAESWTALVKAREAAQAVADNDKATAYDVALALTNLESAIAGLEKTGEEPGPGPVEVNKTDLQTAVNKASKLEKADYTTNSWEAFAEALKAAQQVLDNKNATQQDVDTALSALQDAISKLEAATEPKPNPEPGVVDKAALNATINKAAAINLGLYTDDSANALRAALKKAREVSDNSNATQKQVDAAREALEKAIAALVKRPAAKGDGNVVSNTGSDVATIALAGLLLAGAGAAIAYRRNREQL

SEQ ID NO: 3 - DNA, 5304 bp, Streptococcus pneumoniae R6
  CDS (1)..(5304)
atgaataaaggattatttgaaaaacgttgtaaatatagtattcggaaattttcattaggtgttgcttctgttatgattggagctacattctttgggacaagtccggttcttgcagatagcgtgcagtctggttccacggcgaacttaccagctgatttagctactgctcttgcaacagcaaaagagaatgatgggcatgattttgaagcgcctaaggtgggagaagaccaaggttctccagaagttacagatggacctaagacagaagaagaactattagcacttgaaaaagaaaaaccggctgaagaaaaaccaaaagaggataaacctgcagctgctaaacctgaaacacctaagacggtaacccctgaatggcaaacggtagagaaaaaagaacaacagggaacagtcactatccgagaagaaaaaggtgtccgctacaaccaattatcctcaactgctcaaaatgataacgcaggtaaaccagccctgtttgaaaagaagggcttgaccgttgatgccaatggaaatgcaactgttgatttaaccttcaaagatgattctgaaaagggcaaatcacgctttggtgtcttcttgaaatttaaagataccaagaataatgtttttgtcggttacgacaaggatggctggttctgggagtataaatctccaacaactagcacttggtatagaggtagtcgtgttgctgctcctgaaacaggatcaacaaaccgtctctctatcactctcaagtcagacggtcagctaaatgccagcaataacgatgtcaatctctttgacacagtgactctaccagctgcggtcaatgaccatcttaaaaatgagaagaagattcttctcaaggcgggctcttatgacgatgagcgaacagttgttagcgttaaaacggataaccaagagggggtaaaaacagaggatacccctgctgaaaaagaaacaggtcctgaagttgatgatagcaaggtgacttatgacacgattcagtctaaggttctcaaagcagtgattgaccaagccttccctcgtgtcaaggaatacagcttgaatggacatactttgccaggacaggttcaacagttcaaccaagtctttatcaataaccaccgaatcacccctgaagtcacttataagaaaatcaatgagacaacagcagagtacttgatgaagcttcgcgatgatgctcacttaatcaatgcggaaatgacagtacgcttgcaagttgtggacaatcaattgcactttgatgtgaccaagattgtcaaccacaatcaagtcactccaggtcaaaagattgatgacgaaagaaaactactttcttctattagtttcctcggcaatgctttagtctctgtttctagtgatcaaactggtgctaagtttgatggggcaaccatgtcaaacaatacgcatgtcagcggagatgatcatatcgatgtaaccaatccaatgaaagatctagccaagggttacatgtatggatttgtttctacagataagcttgctgctggtgtttggagtaactctcaaaacagctatggtggtggttcgaatgactggactcgtttgacagcctataaagaaacagtcggaaatgccaactatgtaggaatccacagctctgaatggcaatgggaaaaagcttataagggcattgttttcccagaatacacgaaggaacttccaagtgctaaggttgttatcactgaagatgccaatgcagacaagaaagtcgattggcaggatggtgccattgcttatcgtagcattatgaacaatcctcaaggttggaaaaaagttaaggatatcacagcttaccgtatcgcgatgaactttggttctcaagcacaaaacccattccttatgaccttggatggtatcaagaaaatcaatctccacacagatggtcttgggcaaggtgttctccttaaaggatatggtagcgaaggccatgactctggtcacttgaactatgctgatattggtaagcgtatcggtggtgtcgaagacttcaagaccctaattgagaaggctaagaaatatggagctcatctaggtatccacgttaacgcttcagaaacttatcctgagtctaaatacttcaatgaaaaaattctccgtaagaatccagatggaagctatagctatggttggaactggctagatcaaggtatcaacattgatgctgcctatgacctagctcatggtcgtttggcacgttgggaagatttgaagaaaaaacttggtgacggtctcgactttatctatgtggacgtttggggtaatggtcaatcaggtgataacggtgcctgggctacccacgttcttgctaaagaaattaacaaacaaggctggcgctttgcgatcgagtggggccatggtggtgagtacgactctaccttccatcactgggcagctgacttgacctacggtggctacaccaataaaggtatcaacagtgccatcacccgctttatacgtaaccaccaaaaagatgcttgggtaggggactacagaagttatggtggtgcagccaactatccactgctaggtggctacagcatgaaagactttgaaggctggcaaggaagaagtgactacaatggctatgtaactaacttatttgcccatgacgtcatgaccaagtacttccaacacttcactgtaagtaaatgggaaaatggtacaccggtgactatgaccgataacggtagcacctataaatggactccagaaatgcgagtggaattggtagatgctgacaataataaagtagttgtaactcgtaagtcaaatgatgtcaatagtccacaatatcgcgaacgtacagtaactctcaacggacgtgtcatccaagatggttcagcttacttgactccttggaactgggatgcaaatggtaagaaactttctactgataaggaaaagatgtactacttcaatacgcaggccggtgcaacaacttggacccttccaagcgattgggcaaagagcaaggtttacctttacaagctaactgaccaaggtaagacagaagagcaagaactaactgtaaaagatggtaaaattaccctagatcttctagcaaatcaaccatacgttctctatcgttcgaaacaaaccaatcctgaaatgtcatggagtgaaggcatgcacatctatgaccaaggatttaacagtggtaccttgaaacattggaccatttcaggcgatgcttctaaggcagaaattgtcaagtctcaaggggcaaacgatatgcttcgtattcaaggaaacaaagaaaaagttagtctcactcagaaattaactggcttgaaaccaaataccaagtatgccgtttatgtcggtgtcgataaccgtagtaatgccaaggcgagcatcactgtaaatactggtgaaaaagaagtgactacttataccaataagtctctcgccctcaactatgtaaaagcctatgcccacaatacacgtcgtaacaatgctacagttgacgatacaagttacttccaaaacatgtacgccttctttacaactggatcggacgtatcaaatgttactctgacattgagtcgtgaagctggtgatgaagcaacttactttgatgaaattcgtacctttgaaaacaattcaagcatgtacggagacaagcatgatacaggtaaaggcaccttcaagcaagactttgaaaatgttgctcagggtatcttcccatttgtagtgggtggtgtcgaaggtgtcgaagacaaccgcactcacttgtctgaaaaacacgatccatatacacaacgtggttggaatggtaagaaagtcgatgatgttatcgaaggaaattggtcactcaagacaaatggactagtgagccgtcgtaacttggtttaccaaactattccgcaaaacttccgttttgaagcaggtaagacctaccgtgtaacctttgaatacgaagcaggttcagacaatacctatgcttttgtagtcggtaagggagaattccagtcaggtcgtcgtggtactcaagcaagcaacttggaaatgcatgaattgccaaatacttggacagattctaagaaagccaagaaggcaaccttcctcgtgacaggtgcagaaacaggggatacttgggtaggtatctactcaactggaaatgcaagtaatactcgtggtgattctggtggaaatgccaacttccgtggttataacgacttcatgatggataatcttcaaatcgaagaaattaccctaacaggtaagatgttgacagaaaatgctctgaagaactacttgccaacggttgccatgactaactacaccaaagagtctatggatgctttgaaagaggcggtctttaacctcagtcaggccgatgatgatatcagtgtggaagaagcgcgtgcagagattgccaagattgaagccttgaagaatgctttggttcagaagaaaacggctttggtagcagatgactttgcaagtcttacagctcctgctcaggctcaagaaggtcttgcaaatgcctttgatggaaacttatctagtttatggcatacatcatggggcggaggagatgtaggcaagcctgcaaccatggtcttgaaagaagcaactgaaatcactggacttcgttatgttccacgtggatcaggttcaaatggtaacttgcgtgatgtgaaacttgttgtgacagatgagtctggcaaggagcatacctttactgcaactgattggccagataacaataagccaaaagacattgattttggtaagacaattaaggctaagaaaattgtccttacaggtactaagacttacggagatggtggcgataaataccaatctgcagcggaactcatctttactcgtccacaggtagcagaaacacctcttgacttgtcaggctatgaagcagctttggctaaggctcagaaattaacagacaaagacaatcaagaggaagtagctagcgttcaggcaagcatgaaatatgcgacggataaccatctcttgacggaaagaatggtggaatactttgcagattatctcaaccaattaaaagattctgctacgaaaccagatgctccaactgtagagaaacctgagtttaaacttagctctgtagcttccgatcaaggtaagacgccagattataagcaagaaatagctagaccagaaacacctgaacaaatcttgccagcaacaggtgagagtcaatttgacacagccctcttcctagcaagtgttagcctagccctatctgctctctttgtagtaaaaacgaagaaagactag

SEQ ID NO: 4 - protein, 1767 aa, Streptococcus pneumoniae R6
MNKGLFEKRCKYSIRKFSLGVASVMIGATFFGTSPVLADSVQSGSTANLPADLATALATAKENDGHDFEAPKVGEDQGSPEVTDGPKTEEELLALEKEKPAEEKPKEDKPAAAKPETPKTVTPEWQTVEKKEQQGTVTIREEKGVRYNQLSSTAQNDNAGKPALFEKKGLTVDANGNATVDLTFKDDSEKGKSRFGVFLKFKDTKNNVFVGYDKDGWFWEYKSPTTSTWYRGSRVAAPETGSTNRLSITLKSDGQLNASNNDVNLFDTVTLPAAVNDHLKNEKKILLKAGSYDDERTVVSVKTDNQEGVKTEDTPAEKETGPEVDDSKVTYDTIQSKVLKAVIDQAFPRVKEYSLNGHTLPGQVQQFNQVFINNHRITPEVTYKKINETTAEYLMKLRDDAHLINAEMTVRLQVVDNQLHFDVTKIVNHNQVTPGQKIDDERKLLSSISFLGNALVSVSSDQTGAKFDGATMSNNTHVSGDDHIDVTNPMKDLAKGYMYGFVSTDKLAAGVWSNSQNSYGGGSNDWTRLTAYKETVGNANYVGIHSSEWQWEKAYKGIVFPEYTKELPSAKVVITEDANADKKVDWQDGAIAYRSIMNNPQGWKKVKDITAYRIAMNFGSQAQNPFLMTLDGIKKINLHTDGLGQGVLLKGYGSEGHDSGHLNYADIGKRIGGVEDFKTLIEKAKKYGAHLGIHVNASETYPESKYFNEKILRKNPDGSYSYGWNWLDQGINIDAAYDLAHGRLARWEDLKKKLGDGLDFIYVDVWGNGQSGDNGAWATHVLAKEINKQGWRFAIEWGHGGEYDSTFHHWAADLTYGGYTNKGINSAITRFIRNHQKDAWVGDYRSYGGAANYPLLGGYSMKDFEGWQGRSDYNGYVTNLFAHDVMTKYFQHFTVSKWENGTPVTMTDNGSTYKWTPEMRVELVDADNNKVVVTRKSNDVNSPQYRERTVTLNGRVIQDGSAYLTPWNWDANGKKLSTDKEKMYYFNTQAGATTWTLPSDWAKSKVYLYKLTDQGKTEEQELTVKDGKITLDLLANQPYVLYRSKQTNPEMSWSEGMHIYDQGFNSGTLKHWTISGDASKAEIVKSQGANDMLRIQGNKEKVSLTQKLTGLKPNTKYAVYVGVDNRSNAKASITVNTGEKEVTTYTNKSLALNYVKAYAHNTRRNNATVDDTSYFQNMYAFFTTGSDVSNVTLTLSREAGDEATYFDEIRTFENNSSMYGDKHDTGKGTFKQDFENVAQGIFPFVVGGVEGVEDNRTHLSEKHDPYTQRGWNGKKVDDVIEGNWSLKTNGLVSRRNLVYQTIPQNFRFEAGKTYRVTFEYEAGSDNTYAFVVGKGEFQSGRRGTQASNLEMHELPNTWTDSKKAKKATFLVTGAETGDTWVGIYSTGNASNTRGDSGGNANFRGYNDFMMDNLQIEEITLTGKMLTENALKNYLPTVAMTNYTKESMDALKEAVFNLSQADDDISVEEARAEIAKIEALKNALVQKKTALVADDFASLTAPAQAQEGLANAFDGNLSSLWHTSWGGGDVGKPATMVLKEATEITGLRYVPRGSGSNGNLRDVKLVVTDESGKEHTFTATDWPDNNKPKDIDFGKTIKAKKIVLTGTKTYGDGGDKYQSAAELIFTRPQVAETPLDLSGYEAALAKAQKLTDKDNQEEVASVQASMKYATDNHLLTERMVEYFADYLNQLKDSATKPDAPTVEKPEFKLSSVASDQGKTPDYKQEIARPETPEQILPATGESQFDTALFLASVSLALSALFVVKTKKD

SEQ ID NO: 5 - DNA, 5901 bp, Bifidobacterium longum NCC2705
  CDS (1)..(5901)
atgaaaaagaagaagactatatcggctgcgctggcaacagcgttagccttaacctgcatgggcagcgggggaggtactgcgttcgcagtgcccctgtctgatgctgacttgcagactttggcaagtcagattcagcaaatcaacgatacttctgattctgcaactgcttccgagactccttccgcacaagccgatgcggttgaaggctggactattgattccaacatcgctcagggcgacgaaatcctggagatggcaaacggttggctgcacctcaagtccactgcctctaacggtaatgcggcagcgaaccccagctccagcaacaactggccggcagtagccgtatggggcacagattacgacttctccaaggccggctccttccacgccaccatcaaatccccgcaggaaggctccgccaaccgcttcggcttctacctgggctacaacgacccgggcagcggcctgttcatcggctacgattcggacggctggttctggcagacctacaccggtggcggtagcggcagctggtacagcggtgctcgtatcgctgctccgagcgccaacgaagagcacgacattcaggtctcctggaccgacgccaaggtcgccacactgaccgtggatggccagaaggcattcgatgtcgattactccgcaatgacgaacctctccaacaagcttgccatcaaggccggctcctggaaggggctgaaccaggtcaccgacgtgtacatcaaggacttcccggaggttgtcgaagccgccaagcacgcggtttccggcaaggttgtggacgctggaggcgctgccattgaaggcgcaacagtgcgtttggataagaccaaggtgaagaccggtgcagatggcaccttctccttcgccgacattgaagaaggcgaacacacgctttcgattgccaaggaaggctatgaggacgtctcccagcaggtggcggtctccggcgcggaccttgccatcgatcccatcaccctgaacaaaaccgttcaggtcgcctccgaaacgctgaagaccaagaagatggaagttcagattaagaagaacttcccctctgtgctgcagtacacgatgaccgacggcaaggtgatgtacggccaaaccaaggatgtgcgcaccgttgagatcaacggcaccaacatcgaactgaccgacgatgacgtgaccttcaagaaggtttccgataccgaagccacctacacgctgaaggtcaaggatgaggccaagaagattgacgcggtgatcaccgttcagatcacggtcaaggccaaccagctgcacctcaacgtcaccaagatcaagaacaacctgtccgaaggcattcctgagggcaacggtgtggaggagaacgccatccagacgctgtccttcccgaaccagagtctcgtttccgtgcgttccagccaggaaaatgcccaattcactggtgctcgtatgtcttccaacacgcagaagcctggcgataccaacttcgcagtgaccgaagatactaacgtcaccgatagcgactacacctacggcttcatctccggtgctggcctgagtgccggcctgtggagcaactccgagcacgatggcacctatgtggcggctcctgtgcgcggcggcagccagaacacgcgtgtctacgccaccacccagcagactggtgacgccacctccctgggcctggccagcgctccgtggtactaccaccgcacggtcaccgattccaagggcaagaagtacaccgtggccgaaaccgctctgccgcagatggccgtggccatcgccggcgacgagaacggtgacggtgccgtcaactggcaggatggcgcaatcgcctaccgcgacatcatgaacaacccgtacaagtccgaggaagttcccgaactggtggcatggcgtatcgccatgaacttcggctcccaggcgcagaacccgttcctcaccacgcttgacaacgtcaagaaggtggccttgaacaccgacggcctcggccagtccgtgctgctcaagggctacggcaatgaaggccacgactccggccacccggactacggcgatatcggccagcgtctcggcggcgccgacgacatgaacaccatgatggaagagggctccaagtatggcgctcgcttcggtgtgcacgtcaacgcctccgaaatgtatccggaagccaaggccttcagcgaggacatggtgcgccgcaactctgcaggcggcctgagctacggctggaactggcttgatcagggtgtcggcatcgacggcatctacgatctggcatccggttctcgtgtaagccgtttcgctgacctcagcaaggaagtcggcgacaacatggacttcatctacctcgatgtgtggggcaacctgacttcttccggttcggaagattcttgggaaacccgcaagatgagcaagatgatcaacgacaacggctggcgtatgaccaccgaatggggttccggcaacgagtacgactccaccttccagcactgggcagctgatctgacctacggcggctacacctccaagggcgagaactccgaagtgatgcgcttcctgcgcaaccaccagaaggacagctgggttggcgactacccgcaatacggcggcgctgccaacgccccgctgctcggcggctacaacatgaaggacttcgaaggctggcagggccgcaacgactatgccgcctacatcaagaacctgtacacccatgatgtgtccactaagttcatccagcacttcaaggtgacccgctgggtcaacaacccgctgctgaccgccgacaatggcaatgccgctgccgtgtccgacccgaacacgaacaacggcaacgagcagattaccctgaaggattccaacggcaacgttgtagtagtctcccgtggttccaacgacacctctagcgcagcctaccgccagcgcaccatcaccttcaacggcgtgaaggtcgcatccggtgtggtctccgcaggcgatggcagcgccactggcgacgagtcctacctgctgccgtggatgtgggattccttcaccggcaagctggtcaaggattccgagcagaagctctaccactggaacaccaagggcggcaccaccacctggacgctgccggacagctggaagaacctctccagcgtaaaggtgtaccagctcaccgatcagggcaagaccaacgagcagaccgttgccgtctccggcggcaaggtgacgcttaccgctgatgccgaaaccccgtacgtggtgtacaagggcgaagccaagcagatccaggtcaactggagcgaaggcatgcatgtggtagacgccggcttcaacggcggctccaacaccctcaccgacaactggaccgtcgccggaaccggcaaggccgaagttgaaggcgacaacaacgccatgctgcgcctgaccggcaaggtcgatgtctcccagcgtctgaccgatctcaaggctggccagaagtacgcgctgtatgttggcgtcgacaaccgctccaccggcgatgcatccgtcaccgtaaccagcggcggcaaggtgctggccgccaactccaccggcaagtccatcgccaagaactacatcaaggcatacggccacaacacgaacagcaatacggaaaatggctccagctacttccagaacatgtacgtgttcttcaccgcgcctgagaacggcgatgccacggtaaccctgtctcacaagagcaccgacggagcacacacctacttcgacgatgtgcgcatcgtggagaaccagtactccggcatcacctatgagaaggacggcacgctgaagtccctcaccaacggattcgaaaacaacgcccagggcatctggccgttcgtggtctccggttccgaaggcgttgaggacaaccgcatccacctctccgagctgcatgctccgttcacgcaggccggttgggatgtcaagaagatggacgatgtgctcgatggcacttggtctgtgaaggttaacggcctgacccagaagggcacgctggtctaccagacgatcccgcagaacgtgaagttcgaggcgggtgccaagtacaaggtgagcttcgactaccagtccggttccgatgacatctacgccatcgctgtgggccagggtgaatactctgccggcagcgtgaagctgaccaacctgaagaaggctctgggtgagaccggcaaggccgagttcgagctgaccggtggcgtcaacggcgattcctggttcggtatttactcgaccgcaaccgcacctgatctgcagggttccaccggcaatgcacaggacttcggcggatacaaggacttcgtgctcgacaacctgaagatcgagcgcatcgagtcccagacccgcaccaaggccgaagcgcaggacaaggtcaaggaaatccgcggcaagtacgattccaagcgtgctgagctctccgatgccgcatggcagcagtatcaggacaccttggtcaaggctcgcgtgctcatcaacaagaatggcgcaaccgctgaggacttcaccaaggcatacgacattctcgtggccctcgacgagtacatgaagaccgctcccggcaacgagagcagcgacaagtacgacgtggcagctgacggctccgatgagctgggtggctacaccgtggccacgggcagcgaagagcctaccgcaggcctgccgagcgaaggcccggccgatctggcacaggatggcaacgacagcacccactggcacaccagctggagcgagaacgcagtcggcaacggcaccgcatggtatcagttcaacctcaacgaaccgaccaccatcaacggcctgcgctacctgccgcgctccggaggtatgaacgccaacggcaagatcaagggctacaagatcacgctcactctggcggatggcaccaccaaggatgtcgtcaccgatgctgagttctccaccaccaccatgtggcagaaggccagcttcgacgccgtcgagaatgtgaccgccgtacgcttgaccgtcctgtcttccgcaggccagagcgactcccaggccaacaagttcgcatccgctgccgaactgcgtttgaccacggaccgcgaggttgaggaagagactgtcgctccggacaagaccgacctcaacgacaccatcgccaaggctaacggtcttaaggaatccgactacacggctgaaagctggactgctctggtcaaggcccgcgaagctgcacaggccgtggcggataacgataaggccaccgcttacgatgtggctctggcgctgacgaacctcgaatccgctatcgctggtctcgagaagaccggtgaggagcctggcccaggcccggttgaggtgaacaagaccgacctgcagactgcagtgaacaaggcaagcaagctcgagaaggccgattacacgaccaactcgtgggaagctttcgccgaggcactgaaggctgcacagcaggtgctcgacaacaagaacgccacccagcaggatgtggataccgcactgagcgctcttcaggacgccatctccaagctggaagctaccgccgagccgaagccgaatccggaaccgggcgtggtggacaaggctgctctgaacgcgaccatcaacaaggccgccgccatcaacctgggtctctacaccgacgactccgccaacgctctgcgcgccgcgctgaagaaggcccgtgaggtctccgacaacagcaacgccacgcagaagcaggtcgacgcagctcgcgaggctctcgagaaggcaattgccggcttggtgaagcgtaccgctgccaagggtgatggcaacgttgtttccaacacgggctccgatgtcgccacgatcgctctggctggactgctgctggcaggtgctggcgccgccatcgcctaccgtcgcaatcgcgagcaactgtga

SEQ ID NO: 6 - protein, 1966 aa, Bifidobacterium longum NCC2705
MKKKKTISAALATALALTCMGSGGGTAFAVPLSDADLQTLASQIQQINDTSDSATASETPSAQADAVEGWTIDSNIAQGDEILEMANGWLHLKSTASNGNAAANPSSSNNWPAVAVWGTDYDFSKAGSFHATIKSPQEGSANRFGFYLGYNDPGSGLFIGYDSDGWFWQTYTGGGSGSWYSGARIAAPSANEEHDIQVSWTDAKVATLTVDGQKAFDVDYSAMTNLSNKLAIKAGSWKGLNQVTDVYIKDFPEVVEAAKHAVSGKVVDAGGAAIEGATVRLDKTKVKTGADGTFSFADIEEGEHTLSIAKEGYEDVSQQVAVSGADLAIDPITLNKTVQVASETLKTKKMEVQIKKNFPSVLQYTMTDGKVMYGQTKDVRTVEINGTNIELTDDDVTFKKVSDTEATYTLKVKDEAKKIDAVITVQITVKANQLHLNVTKIKNNLSEGIPEGNGVEENAIQTLSFPNQSLVSVRSSQENAQFTGARMSSNTQKPGDTNFAVTEDTNVTDSDYTYGFISGAGLSAGLWSNSEHDGTYVAAPVRGGSQNTRVYATTQQTGDATSLGLASAPWYYHRTVTDSKGKKYTVAETALPQMAVAIAGDENGDGAVNWQDGAIAYRDIMNNPYKSEEVPELVAWRIAMNFGSQAQNPFLTTLDNVKKVALNTDGLGQSVLLKGYGNEGHDSGHPDYGDIGQRLGGADDMNTMMEEGSKYGARFGVHVNASEMYPEAKAFSEDMVRRNSAGGLSYGWNWLDQGVGIDGIYDLASGSRVSRFADLSKEVGDNMDFIYLDVWGNLTSSGSEDSWETRKMSKMINDNGWRMTTEWGSGNEYDSTFQHWAADLTYGGYTSKGENSEVMRFLRNHQKDSWVGDYPQYGGAANAPLLGGYNMKDFEGWQGRNDYAAYIKNLYTHDVSTKFIQHFKVTRWVNNPLLTADNGNAAAVSDPNTNNGNEQITLKDSNGNVVVVSRGSNDTSSAAYRQRTITFNGVKVASGVVSAGDGSATGDESYLLPWMWDSFTGKLVKDSEQKLYHWNTKGGTTTWTLPDSWKNLSSVKVYQLTDQGKTNEQTVAVSGGKVTLTADAETPYVVYKGEAKQIQVNWSEGMHVVDAGFNGGSNTLTDNWTVAGTGKAEVEGDNNAMLRLTGKVDVSQRLTDLKAGQKYALYVGVDNRSTGDASVTVTSGGKVLAANSTGKSIAKNYIKAYGHNTNSNTENGSSYFQNMYVFFTAPENGDATVTLSHKSTDGAHTYFDDVRIVENQYSGITYEKDGTLKSLTNGFENNAQGIWPFVVSGSEGVEDNRIHLSELHAPFTQAGWDVKKMDDVLDGTWSVKVNGLTQKGTLVYQTIPQNVKFEAGAKYKVSFDYQSGSDDIYAIAVGQGEYSAGSVKLTNLKKALGETGKAEFELTGGVNGDSWFGIYSTATAPDLQGSTGNAQDFGGYKDFVLDNLKIERIESQTRTKAEAQDKVKEIRGKYDSKRAELSDAAWQQYQDTLVKARVLINKNGATAEDFTKAYDILVALDEYMKTAPGNESSDKYDVAADGSDELGGYTVATGSEEPTAGLPSEGPADLAQDGNDSTHWHTSWSENAVGNGTAWYQFNLNEPTTINGLRYLPRSGGMNANGKIKGYKITLTLADGTTKDVVTDAEFSTTTMWQKASFDAVENVTAVRLTVLSSAGQSDSQANKFASAAELRLTTDREVEEETVAPDKTDLNDTIAKANGLKESDYTAESWTALVKAREAAQAVADNDKATAYDVALALTNLESAIAGLEKTGEEPGPGPVEVNKTDLQTAVNKASKLEKADYTTNSWEAFAEALKAAQQVLDNKNATQQDVDTALSALQDAISKLEATAEPKPNPEPGVVDKAALNATINKAAAINLGLYTDDSANALRAALKKAREVSDNSNATQKQVDAAREALEKAIAGLVKRTAAKGDGNVVSNTGSDVATIALAGLLLAGAGAAIAYRRNREQL

SEQ ID NO: 7 - DNA, 5061 bp, Clostridium perfringens str. 13
  CDS (1)..(5061)
atgggtagaaaatgcatgaataagaagattgccgcaataatagcagctgcagttattgtaggacaattaccaatttcagtacttgctacacctgttaatgaagctggagatgagattaatagtgaatcagctgaaattttaactaatagtgatgaagaagctgaggcttatattcaaaactatgatagaccagaggggattacttggacaaaattagcaggctcaggaagtgttgaggtaactgatggatttttatcagttactaataatggagattatagaattatggaagatcaatcgcctaatataaaaaatggtgagttagaaagtaaatttacagttggaggttctcaaactggaataatatttagagcaactgagtcaaactatggaatgatcaactataactcaggtactggctgggttatagaaaataaaaatagttgggaggatattacaggaccaaaactaaataatggagatgttgtaacagttaaggctacttttgttgaaaagcatttaactgttaatgtttctgtaaatgatggagaatttgaaactatatatgataaagaatcagatttaattccattacaagctggtaaagttggatatagaggatggggtaacgctaaaactaccaaatttgattatattaagtatgctcctatgactatagataagggacctatagtatcaataaatgaagtaaatgtagaaacttatccaagagttaagcctattttaccatcaagtgttacagtaaatcatgaaaatggtatgtctagtattaaggatgtttcttggaattatatacctaaggaaagttattcaaagccaggtacattcaaagttgaaggtacagttgaaggtacagatgtaaaagcaatagctaatgtaactgtaagttcagatttagcatattatgaaactaattttgaaacagaagaaacaagaggagattggcaagttgtacaaggaggaggatctccaagttatgaagagggtaaagtaaaaatacctatgaatggagtatcaatcgctgttgatatgaattctccagaggttaagaactttacttatgaaactgatttttctgttgataataatggaggaagaataggtctattatttagatatgtatcagaaactgagtggggagctgtttgctatgataatggttcttgggtatggaaaactggagatggcaaatatggtaatttcccaggaacatttacaccagagccaggaaaaacttacagaataaagcttaaagtagaagatacaaatattactatgtgggttgatggagagaaaatagggcaagttgcagtatctaatttaccagatgtaagaggaaaagttggcttaactggatggtttggaaataaaaatgttactttagataatcttgttgttgaggaattaggtggaataatggcaccagaagtaggtccattagaagaacaaagtatagaatcagattctatgaaagttgttttagataatagattcccaactgttattagatatgagtggaaaggaactgaagatgttttatcaggagcatctgtagatgatttagaagctcaatatatggttgaaattaatggtgaaaagagaattccaaaagtaactagtgaatttgcaaataatgaaggtatatatacattaaactttgaagatataggaatgactattactttaaagatgactgttaatgaaaataaattaagaatggaagttactgatattcaagaaggggatgttaaacttcaaacattaaatttcccaaatcatagtttagcttcagtaagcagtttaaataatggtaaaacagcctctgttctaacaactggtgactggaataacataaatgaagagtttacagatgttgctaaggcaaaaccaggggttaagggtaaaacttatgcatttataaatgatgataagtttgctgttactataaataataatactattgaaggtggaaatagggttgtattaacaacagaaaatgatactcttcctgataatacaaactataagaaagttggaatatcaaacggtacttggacttataaagaaatacttcaagatacaacagatcaaggaagtaagctatatcaaggtgaaaagccatggtcagaagtaattatagcaagagatgagaatgaagatggtcaagtagattggcaagatggtgctattcaatatagaaaaaatatgaaaattccagtaggtggagaagaaataaaaaatcaaatgtcatatattgattttaatattggatacactcaaaatccattcttaagatcattagatacaattaaaaagctttcaaattatacagatggatttggacaattagttcttcataagggatatcaaggagaaggacatgacgactctcatccagactatggcggacatattggtatgagacaaggtggtaaggaagacttcaataccttaatagaacaaggtaaggaatataatgctaaaataggtgttcatataaatgcaaccgaatatactatggatgcgtttgaatatccaactgaattagttaacgaaaatgctccaggatggggatggttagaccaagcttattatgtaaatcaaagaggagatataactagtggtgaattattcagaagacttgatatgttaatggaagatgcaccagaactaggatggatttacgttgacgtttatacaggtaatggatggaatgctcatcaattaggagaaaagataaatgattatggaattatgattgcaacagaaatgaatggaccattagagcaacatgttccatggactcactggggtggagatcctgcatatccaaacaagggaaatgcaagtaaaataatgagatttatgaaaaatgatactcaagattcattcttagcagatccattagtaaaaggtaataagcatttattatcaggtggatggggaactagacatgatatagaaggtgcttatggtacagaagtattctataatcaagtattacctactaagtatttacaacacttccaaataactaagatgagcgaaaatgaagtattatttgaaaatggagttaaggctgttagagaaaactctaatataaattactatagaaatgatagattagttgctactactccagaaaattcaattggaaatactggtataggggatactcaattattcttaccatggaatcctgttgatgaggcaaatagtgaaaagatatatcactggaatccattaggaactacttcagaatggactttaccagaaggatggacttctaatgacaaagtttatttatatgaattatcagatttaggaagaactttagttaaggaagtaccagttgtagatggaaaagttaatttagaagtaaaacaagatactccttatatagttactaaggaaaaagttgaagagaagagaatagaggattggggatatggctcagaaatagctgatccaggatttgattctcaaacttttgataagtggaataaagaatctactgctgaaaatacagatcatataactattgaaaatgaaagtgttcaaaagagattaggtaatgatgttcttaaaataagtggaaatgaaggcgctgatgctaaaatttctcaaagcataagtggattagaagaaggagtaacttattcagtatctgcatgggttaaaaatgataataatagagaagttacactaggagttaatgttggtggaaaagatttcactaatgtaataacaagtggtggtaaagtaagacaaggagaaggtgttaagtatattgacgatactttcgtgagaatggaagtagaatttactgtacctaaaggagtaaattcagctgatgtttacttaaaggcttcagaaggagatgctgactcagttgtactagttgatgactttagaatatgggatcatccaggacacactaatagagatggatatgtattctacgaagattttgaaaatgtagatgaaggtatatcaccattttatttatctccaggaagaggacattcaaatagatctcacttagctgagaaggatatatctatagatgctaatcaaagaatgaactgggtacttgatggaagattctcattaaaatctaaccaacaaccaaaggaaataggtgaaatgttgactacggatgtttcatcattcaaattagaaccaaataaaacttatgaatttggattcttatattcattagaaaatgctgctccaggatattctgtaaatattaagaatagagatggtgaaaagattgtaagtattcctttagaggctactggttcaaattatgcacaagatattttcactaaaactaaatctgtaactcacgagtttacaactggagattttgctggagattactatattactttagaaaaaggtgatggatttaaagaagtaatcttagataatatttatgttaaggaaatagataagtcaattgaatcacctgaattagctcatgtaaacttaaatacagtagaacatgacttagaagttggacaaagcgttccatttgctataaatgcattaatgaataatggtgcaaatgttaacttagaagaagctgaagttgaatataaagtttcaaaaccagaagttttaactattgaaaatggaatgatgactggagcttctgaaggctttactgatgtccaagtaaatattactgttaatggaaataaagtttcttcaaatacagtaagagttaaggttggaaatccagaagttgaggaagaagaagttatagtaaacccagtaagaaactttaaggttactgataagactaagaagaatgtaactgtaagctgggaagagccagaaaaaacttatggattagaaggctatgttctttataaagatggtaagaaagttaaagaaataggtgctgataaaactgaatttacatttaagggattaaacagacacactatttataactttaagattgctgctaaatattctaatggtgaactttcaactaaggaatcaataactgttagaactgcaagataa

SEQ ID NO: 8 - protein, 1686 aa, Clostridium perfringens str. 13
MGRKCMNKKIAAIIAAAVIVGQLPISVLATPVNEAGDEINSESAEILTNSDEEAEAYIQNYDRPEGITWTKLAGSGSVEVTDGFLSVTNNGDYRIMEDQSPNIKNGELESKFTVGGSQTGIIFRATESNYGMINYNSGTGWVIENKNSWEDITGPKLNNGDVVTVKATFVEKHLTVNVSVNDGEFETIYDKESDLIPLQAGKVGYRGWGNAKTTKFDYIKYAPMTIDKGPIVSINEVNVETYPRVKPILPSSVTVNHENGMSSIKDVSWNYIPKESYSKPGTFKVEGTVEGTDVKAIANVTVSSDLAYYETNFETEETRGDWQVVQGGGSPSYEEGKVKIPMNGVSIAVDMNSPEVKNFTYETDFSVDNNGGRIGLLFRYVSETEWGAVCYDNGSWVWKTGDGKYGNFPGTFTPEPGKTYRIKLKVEDTNITMWVDGEKIGQVAVSNLPDVRGKVGLTGWFGNKNVTLDNLVVEELGGIMAPEVGPLEEQSIESDSMKVVLDNRFPTVIRYEWKGTEDVLSGASVDDLEAQYMVEINGEKRIPKVTSEFANNEGIYTLNFEDIGMTITLKMTVNENKLRMEVTDIQEGDVKLQTLNFPNHSLASVSSLNNGKTASVLTTGDWNNINEEFTDVAKAKPGVKGKTYAFINDDKFAVTINNNTIEGGNRVVLTTENDTLPDNTNYKKVGISNGTWTYKEILQDTTDQGSKLYQGEKPWSEVIIARDENEDGQVDWQDGAIQYRKNMKIPVGGEEIKNQMSYIDFNIGYTQNPFLRSLDTIKKLSNYTDGFGQLVLHKGYQGEGHDDSHPDYGGHIGMRQGGKEDFNTLIEQGKEYNAKIGVHINATEYTMDAFEYPTELVNENAPGWGWLDQAYYVNQRGDITSGELFRRLDMLMEDAPELGWIYVDVYTGNGWNAHQLGEKINDYGIMIATEMNGPLEQHVPWTHWGGDPAYPNKGNASKIMRFMKNDTQDSFLADPLVKGNKHLLSGGWGTRHDIEGAYGTEVFYNQVLPTKYLQHFQITKMSENEVLFENGVKAVRENSNINYYRNDRLVATTPENSIGNTGIGDTQLFLPWNPVDEANSEKIYHWNPLGTTSEWTLPEGWTSNDKVYLYELSDLGRTLVKEVPVVDGKVNLEVKQDTPYIVTKEKVEEKRIEDWGYGSEIADPGFDSQTFDKWNKESTAENTDHITIENESVQKRLGNDVLKISGNEGADAKISQSISGLEEGVTYSVSAWVKNDNNREVTLGVNVGGKDFTNVITSGGKVRQGEGVKYIDDTFVRMEVEFTVPKGVNSADVYLKASEGDADSVVLVDDFRIWDHPGHTNRDGYVFYEDFENVDEGISPFYLSPGRGHSNRSHLAEKDISIDANQRMNWVLDGRFSLKSNQQPKEIGEMLTTDVSSFKLEPNKTYEFGFLYSLENAAPGYSVNIKNRDGEKIVSIPLEATGSNYAQDIFTKTKSVTHEFTTGDFAGDYYITLEKGDGFKEVILDNIYVKEIDKSIESPELAHVNLNTVEHDLEVGQSVPFAINALMNNGANVNLEEAEVEYKVSKPEVLTIENGMMTGASEGFTDVQVNITVNGNKVSSNTVRVKVGNPEVEEEEVIVNPVRNFKVTDKTKKNVTVSWEEPEKTYGLEGYVLYKDGKKVKEIGADKTEFTFKGLNRHTIYNFKIAAKYSNGELSTKESITVRTAR

SEQ ID NO: 9 - DNA, 4086 bp, Streptomyces coelicolor A3(2)
  CDS (1)..(4086)
atggcagcgatcacaccgaagcgatcggtcagagtggacacctcgacaggctcatcatctcccccaaccggacgcgcacgggtccggcggcacggcgccgtcgtcgcggcgctgggcctcaccgcgggcctgctgtcggccgccgccctgcccgcgggcgcggccccgccccgccccgctgccgctgcggcgcccgcgggggcaccgacgccggtggaactgagccggggcgggctgaccgtcaccgtggcgaaggagttcccccaggtgatctcctaccggctgggccggcgcggactcgacgggcgggcaacggcgctggacggcttcaccgtcaacggcgagagccaccgcgccaccaccaccgtgaaggcgaagggcagcagggcggtctacacctccacgttcgaggacctgcccggtctcacgatcacctccagcatcacggtcaccaaggagacgacggtcgtcttcgccgtcgagaagatctcgggcgaggccgcgccgggcgtgcgcaccctcgcgatccccggccagtccctcgtctccgtcgactccgccgagccgggcgccaacctcgcccggacgaagatctccaccgactcgacgacgaccgccgaccgcttcgtccccgtcaccggcgacaccgccccggacaaggggcccgtcggcaccccgtacgcgttcgtcggcaacgcgcagttgtcggcgggcatcatcaccaacgcgaccgaggactcgccgcaggacgacaacaccgactggaacacccgcctgcagtcccgcatcgtcgacgagggcgagggacggcgccgggcggagctgtcggccggcacgtacacctaccaccccgagggcgccaccgatccacgggtcgacacctacgagctgccccgggcgacggtggtcctcgccgccgacgccaaccgggacggcacggtcgactggcaggacggcgccatcgcccaccgggagcacatgcgccgcccgctcggcgccgaccgggtgcccgaacgcgtggtccagcgcatcccgttcaacttcgcgagccaggccaccaacccgttcctgaagacgctggacaacaccaagcgcatctccatggccaccgacgacctcgggcagtgggtgctggagaaggggtacgcgagcgagggccacgactccgcccaccccgactacggcggcaacgagaacgtccgcgcgggcggctggaaggacctgaaccgcctcacccggacgggagccggctacaacgcggacttcgccgtccacgtcaacgccacggaggcctacgcccaggccaggaccttcaccgaggacatggtcgcgggccaggccgacggctgggactggctcaaccaggcctaccacatcgaccagcgcaaggacctgggcaccggcgccgtcctcgaccgcttcaagcagctccgcaaggaagcaccgggcatcagaaccgtctacatcgacgcctactactccagcggctggctggccgacggtctggccgccgggctgcgtgagatgggcttcgaggtcgccaccgagtgggcgtacaagttcgagggcacctcggtgtggtcgcactgggcggccgacaagaactacggcggcgccacgaacaagggcatcaactcggacatcgtccggttcatcgccaacgccgaccgcgacgtgtggaacgtggacccgctgctcggcggcgcgagcgtcgtcgagttcgagggctggaccggccaggacgactggaacgccttctaccgcaacatctggaccgacaacctgccgaccaagttcctccagcacttccaggtgctggactgggaccgcggcaggtccgcgaggctcaccggcggggtggacgtgaagtccgtcgacggcgagcggcggatctccatggacggcaccgaggtcctcaagggcgacacctacctgctgccctggcagaacgccgggaaggacgacggcacctcgtcgccccgcgacgccgacaagatgtacttctacagcgcctccggcggcgagcacaccttcgaactgaccggacagttcgcgggcaccgaggacttcaccctctacgaactcaccgaccagggccgggcggagaaggcccgggtgacggcccacgaggggcgggtgaccctcaccgccgagaaggggcagccctacgtcctcgtgccgaacggcggcagggcaccgcaccgcgacgcccactacggcgagttcaccgggctgtccgaccccggcttcaacggcggggacctcgacgcctggaacgcgagcggcggcgcggagatcgtccgggccggcaacggggacaacgtggtccgcctgggcgaggacgcctcgggcatcgcgcagcgggtccgcggcctgaccccgggcgagcggtacacgctcggcgcggacgtcgggatcggccccggcgagcgccgggagacgacgctgcgggtgcgcggcggcaaggacagcgaggccaggaccttcgacatcacgccggcgcggaacaggatggcgtccgacgagaagcgagacacgtactcccagcgggcctcggtctccttcaccgcgccgcgcgacggctcggtcaccgtggagctcggggcggtcgccggtggcgccccggtggtcctggacgacgtacgggtcatggtggacaccaccgctccgctcccccgctcccaggacggcacggtcgtcgcccacgacgacttcgagggcaaccggcccggctggggaccgttcgtcaagggcgacgccggcggcgtcaccgacccccgcaccagcatcagcgacctgcacgcgccctacagccagaaggagtggaagaacacctactcgccctacgacaccggcgcgctgaagggcagggccgtcgacgacgtgctcgcgggccggcactcgctgaagtcccacgccgagaacaccgggctggtccaccgcacgacccctgccaccgtgccgttcgaggagggccaccggtaccgggtctccttctcgtaccagaccaacgtcgagggccagtgggcctgggtcaccggcgcggaccgggtcgccgacgggacgacgacctcgcgggacatcacccgtgacgtcctggcacccgccctggacacggcggcctactcccgcgagttcgtcgccggctgcggggacacctgggtcgggctgcgcagactcggcagcgcccggggcaccgatctcgtcctcgacgacttcacggtgaccgacctcggcgaggcggacaccggggccgcctgcgccgctgtcacggccccgtccggcgccgaactgagccccggggtccccggtgagtacgtcacggcgttcaccaaccacgagtccgccggcgccgagaacgtgggcatcgccctccagggtctgcccgagggctggaaggccgaggtgaaggagaaggacggcaatctcttcgagcgcgtgcagccgggcgcgaccgtgcgcaccacctggctcctcaccccgcccgccggcacggccggcacctccgccacgtggcaggtgacggccgcctacgcacacgagggagccaccaggacggtctccaccggggcccgcgccgcggtgaccgacgaaccggtactggccccggcttcgacgaccgcgaccgccgactcggagaacacctcgtcgggcgcctccgaagggccggtgtccaacgtcctcgacggtgacgccggcaccatctggcacaccgactacaccacctcccaggcgccgtatccgcactgggtgacgctgaagctcggcggcgccgccgacgtcgacgggttcgggtacctgggccgacagagcgggggcccgaacggacgcgtcgccgactacgaggtcgccgtgtcggacgacggcgaggcgtggacgacggtggccaccggcaccctgaaggacgtcccgcggacccagcgcgtctccttcgaccgggtccgcgcctcctacgtgcgcttcaccgccctcgacgcgctcaacgggcagccctacgcggccgccgcggagatgcgcgtgtacggcgtgcccgtggacctgcccacgggctacccgccgggcgaacgccccgccgacgcccgctga

SEQ ID NO: 10 - protein, 1361 aa, Streptomyces coelicolor A3(2)
MAAITPKRSVRVDTSTGSSSPPTGRARVRRHGAVVAALGLTAGLLSAAALPAGAAPPRPAAAAAPAGAPTPVELSRGGLTVTVAKEFPQVISYRLGRRGLDGRATALDGFTVNGESHRATTTVKAKGSRAVYTSTFEDLPGLTITSSITVTKETTVVFAVEKISGEAAPGVRTLAIPGQSLVSVDSAEPGANLARTKISTDSTTTADRFVPVTGDTAPDKGPVGTPYAFVGNAQLSAGIITNATEDSPQDDNTDWNTRLQSRIVDEGEGRRRAELSAGTYTYHPEGATDPRVDTYELPRATVVLAADANRDGTVDWQDGAIAHREHMRRPLGADRVPERVVQRIPFNFASQATNPFLKTLDNTKRISMATDDLGQWVLEKGYASEGHDSAHPDYGGNENVRAGGWKDLNRLTRTGAGYNADFAVHVNATEAYAQARTFTEDMVAGQADGWDWLNQAYHIDQRKDLGTGAVLDRFKQLRKEAPGIRTVYIDAYYSSGWLADGLAAGLREMGFEVATEWAYKFEGTSVWSHWAADKNYGGATNKGINSDIVRFIANADRDVWNVDPLLGGASVVEFEGWTGQDDWNAFYRNIWTDNLPTKFLQHFQVLDWDRGRSARLTGGVDVKSVDGERRISMDGTEVLKGDTYLLPWQNAGKDDGTSSPRDADKMYFYSASGGEHTFELTGQFAGTEDFTLYELTDQGRAEKARVTAHEGRVTLTAEKGQPYVLVPNGGRAPHRDAHYGEFTGLSDPGFNGGDLDAWNASGGAEIVRAGNGDNVVRLGEDASGIAQRVRGLTPGERYTLGADVGIGPGERRETTLRVRGGKDSEARTFDITPARNRMASDEKRDTYSQRASVSFTAPRDGSVTVELGAVAGGAPVVLDDVRVMVDTTAPLPRSQDGTVVAHDDFEGNRPGWGPFVKGDAGGVTDPRTSISDLHAPYSQKEWKNTYSPYDTGALKGRAVDDVLAGRHSLKSHAENTGLVHRTTPATVPFEEGHRYRVSFSYQTNVEGQWAWVTGADRVADGTTTSRDITRDVLAPALDTAAYSREFVAGCGDTWVGLRRLGSARGTDLVLDDFTVTDLGEADTGAACAAVTAPSGAELSPGVPGEYVTAFTNHESAGAENVGIALQGLPEGWKAEVKEKDGNLFERVQPGATVRTTWLLTPPAGTAGTSATWQVTAAYAHEGATRTVSTGARAAVTDEPVLAPASTTATADSENTSSGASEGPVSNVLDGDAGTIWHTDYTTSQAPYPHWVTLKLGGAADVDGFGYLGRQSGGPNGRVADYEVAVSDDGEAWTTVATGTLKDVPRTQRVSFDRVRASYVRFTALDALNGQPYAAAAEMRVYGVPVDLPTGYPPGERPADAR

SEQ ID NO: 11 - DNA, 3975 bp, Enterococcus faecalis V583
  CDS (1)..(3975)
atgaaacatggaaaaataaaacgatttagtacattgacactattggcaagcgcaacgattttagtaccattaagtacgtctgcagaagaaacaacaaacagcagcactgaaacaagctcatcaatggtggaacctaccgcaacagaagaaaaattgtggcaatctgattttccaggagggaaaactggtgagtggcaagatgtgattggtaaaacaaatcgagaattagcaggagagtcattggcgatttcacgagatgcagcagcaggtaataatgccgtatctttaaatttagattcccctaaattagctgatggtgaagtagaaacaaagtttaaatacactgctggaagcggtcgaacgggtgtgattatacgtggtaataccaaagatagctgggtttttgtaggctataacgcgaatggcaaatggttagttgaaagccctaattcgtggaatgattcaatttctgggccaacgttaaatgaagatacgaattatttgttgaaagtacgttatgttggtgaaaaaattactatctggcttaacaccacgttgatttacgaaggagaacctgttttggccaatggagacaaaattccaacagaagccggacatgtgggtgtccgtttgtggtacgacaaaaaaattgtcaattatgactattttaaaaatggccccgtagatagcattccagaaattgtgccagaagtgacacaaattgcgccagtcaaagtttttacaaaaattggtgtcgcaccaaaattaccgaaacaagtaaaagtgacctataatactggtaaagaagccaatgaagcagtccgttggaatgaaatcgatcctgatgcatataaagaaccaggaacttttgaagtcgacggtactttggaaaatacaaacatcaaagcaaaagccagcattgttgttgctaaagacaatgaagctgaaaaaggcgacaaaatctcttccgcagatttaacagccgtggttgatccacaatttccacgaattattcgctacgaagaccctcagagtaatcaagtgatttttaatggccaacacgagaaaattgaccaagtaatgattgatggcaaagcatataaagcaactgctgaaaaacagaagagtgaagcaaatcaagccgtttataacgtagctgtcccagaaattggtttgcgtttcacaacgacattgactgtttccgaaggccaagaattagctatgaaactctcagatattcgtgaagaaggaaccaaaatacacacaatttcaattccaaatcaaggcttgatttctgtcaatagtacagatgaaggggcgacttttgctggcgttgtgatgaatactgggacaaatgcaaataacggaaataaaaatggtgatactatccaagatttaactacaacaagccaagaagaaacgaaaaaatatatgtatggtttcttaaatacggcgaattatgctgcaagtttttggacgaacgcctatggagacggctctgtcgatggtagtgacaacaatcgaatccataaacaaacaaaagaagcggcgactggttttgtaacaaccttgtcaagtggggcatggacctatcgaccatttgatgcaccggaagattacacaactggagaaacgccagaagtgaaagttaaattctcaaaagatagcaacgacgacaatcgggtggattggcaagatgcggcaattgggttccgttcaattatgaataacccaatgggtgcggaaaaagtccctgaattagtcaaccaacggattccttttaactttgctagtcaggcgacaaacccattcttagtgacgttagacgaatcaaaacgtatttacaatttaacagatggattaggacaaatgaatttactaaaagggtatcaaaatgaaggacatgattctgcgcatccagattacggtgctattggtcagcgacctggtggggaacaagcgttgaatcaattaattgatgaaggacataaattaaatgccgttttcggtgtgcatattaatgacaccgagtcttacccagaagcaaaaggatttaatgaggaattagttgatccaacgaagcgtggctgggattggttagatccgtcttattttattaaacaaagacccgatacattgagtggtcgtcgctatgagcgctttaaagaattaaaacaaaaagcaccgaatctagattatatttatgttgatgtttggggcaaccaaggcgaatcaggttgggcaagtcgtcaactaagtaaagaaattaattcactcggttggtttacaaccaatgaatttccgaatgctttagagtatgactcggtttggaaccattggtctgcagaaaaagattacggcggtacaacaacgaaaggctttaacagtacaatcgttcgttttattcggaatcatcaaaaagacacttggattatttccgacaaccctttgttaggtggagctgaatttgaagcctatgaaggttgggtgggtaaaaccaattttaatacctatcgccaaaaaacttttgccattaatgtcccaactaagttcttacaacattaccaaattacaaactgggaaactacaacagcagcagatggtcaaatctatggcacaattaaattagcgaacggtgctgaaaaagtgaccgttactcaagcagatgctaattcgccaagaagcattacgttaaatgagacagaagttctaaaaggtgatgcgtatctactgccttggaatgtcaatggtcaagacaaactatatcactggaatccaaaaggcggtaccagcacttggtcattggataagaaaatgcaaggaaaaacgaatttacatttatatgaattaacagatcaagggcgtattgacaaaggcgcaattgccactacaaataaccaagtgaccatccaagccgaggctaatacaccgtatgtcattgctgaacctgacagtattgaaccgatgacatttggaacaggaacaccatttaaagatcctggatttaatgaagccaataccttaaaaaataactggaaagttttccgaggtgatggagaggttaaaaaagatgccaatggtgattatgtctttagttcagaaaaagaaagaaccgaaatcaaacaagatatcaatcttcctaaaccaggaaaatatagtttgtatctaaacacagaaacacatgatcgtaaagccacagtaactgttaaaattggtggtaagaaatatacgcggacagtgaataattcggttgcccaaaactacattcaggcagatattaaccatacaagcaggaaaaatccgcagtatatgcaaaatatgcgaattgattttgaaatcccagataatgccaaaaaaggctcggtgacattagcggttgataaaggcaattccgttacaaaatttgatgatttacgaattgttgagcgtcaaacggatatcatgaacccagacaaacaaacagttattaagcaagattttgaagacacacaagcagttgggttatatccgtttgttaaaggctcagctggtggtgtagaagatccacggattcatttatcagaaagaaatgaaccttacacacaatatggttggaatggaaaccttgtttcagatgtattagaaggcaactggtccttgaaagcccataaacaaggagcaggattgatgcttcaaacaattccgcaaaatattaaatttgaaccgaacaagaaatatacggtccaatttgattatcaaactgatggtgaaaatgtctttactgctgggaccattaatggggagttgaaaaataacaatgactttaagccagtcggtgagttaacttcgacagcagcagatggtcaaaccaagcattatgaagcagaaataattggggatgcttcaggaaacactacgtttggtatttttacaacaggtgccgataaagatttcattatggataactttacggtcacagtggaatcaaaaaaataa

SEQ ID NO: 12 - protein, 1324 aa, Enterococcus faecalis V583
MKHGKIKRFSTLTLLASATILVPLSTSAEETTNSSTETSSSMVEPTATEEKLWQSDFPGGKTGEWQDVIGKTNRELAGESLAISRDAAAGNNAVSLNLDSPKLADGEVETKFKYTAGSGRTGVIIRGNTKDSWVFVGYNANGKWLVESPNSWNDSISGPTLNEDTNYLLKVRYVGEKITIWLNTTLIYEGEPVLANGDKIPTEAGHVGVRLWYDKKIVNYDYFKNGPVDSIPEIVPEVTQIAPVKVFTKIGVAPKLPKQVKVTYNTGKEANEAVRWNEIDPDAYKEPGTFEVDGTLENTNIKAKASIVVAKDNEAEKGDKISSADLTAVVDPQFPRIIRYEDPQSNQVIFNGQHEKIDQVMIDGKAYKATAEKQKSEANQAVYNVAVPEIGLRFTTTLTVSEGQELAMKLSDIREEGTKIHTISIPNQGLISVNSTDEGATFAGVVMNTGTNANNGNKNGDTIQDLTTTSQEETKKYMYGFLNTANYAASFWTNAYGDGSVDGSDNNRIHKQTKEAATGFVTTLSSGAWTYRPFDAPEDYTTGETPEVKVKFSKDSNDDNRVDWQDAAIGFRSIMNNPMGAEKVPELVNQRIPFNFASQATNPFLVTLDESKRIYNLTDGLGQMNLLKGYQNEGHDSAHPDYGAIGQRPGGEQALNQLIDEGHKLNAVFGVHINDTESYPEAKGFNEELVDPTKRGWDWLDPSYFIKQRPDTLSGRRYERFKELKQKAPNLDYIYVDVWGNQGESGWASRQLSKEINSLGWFTTNEFPNALEYDSVWNHWSAEKDYGGTTTKGFNSTIVRFIRNHQKDTWIISDNPLLGGAEFEAYEGWVGKTNFNTYRQKTFAINVPTKFLQHYQITNWETTTAADGQIYGTIKLANGAEKVTVTQADANSPRSITLNETEVLKGDAYLLPWNVNGQDKLYHWNPKGGTSTWSLDKKMQGKTNLHLYELTDQGRIDKGAIATTNNQVTIQAEANTPYVIAEPDSIEPMTFGTGTPFKDPGFNEANTLKNNWKVFRGDGEVKKDANGDYVFSSEKERTEIKQDINLPKPGKYSLYLNTETHDRKATVTVKIGGKKYTRTVNNSVAQNYIQADINHTSRKNPQYMQNMRIDFEIPDNAKKGSVTLAVDKGNSVTKFDDLRIVERQTDIMNPDKQTVIKQDFEDTQAVGLYPFVKGSAGGVEDPRIHLSERNEPYTQYGWNGNLVSDVLEGNWSLKAHKQGAGLMLQTIPQNIKFEPNKKYTVQFDYQTDGENVFTAGTINGELKNNNDFKPVGELTSTAADGQTKHYEAEIIGDASGNTTFGIFTTGADKDFIMDNFTVTVESKK

SEQ ID NO: 13 - DNA, 26 bp, Artificial Sequence
  Synthetic DNA
ttaacctccatgggcagcgggggagg

SEQ ID NO: 14 - DNA, 30 bp, Artificial Sequence
  Synthetic DNA
aacctgcggccgccagttgctcgcgattgc

SEQ ID NO: 15 - DNA, 20 bp, Artificial Sequence
  Synthetic DNA
tgcgattcatcgcctagcag

(19) United States
(12) Patent Application Publication
(10) Pub. No.: US 2006/0068461 A1
(43) Pub. Date: Mar. 30, 2006

(54) Transglycosylation method and protein having transglycosylation activity

(75) Inventors: Fusako Oura, Oosaka (JP); Kiyotaka Fujita, Kagoshima (JP); Noriko Nagamine, Kyoto (JP); Takane Katayama, Ishikawa (JP); Hidehiko Kumagai, Ishikawa (JP); Kenji Yamamoto, Shiga (JP)

Correspondence Address: OBLON, SPIVAK, MCCLELLAND, MAIER & NEUSTADT, P.C., 1940 DUKE STREET, ALEXANDRIA, VA, 22314, US

(73) Assignee: Ajinomoto Co., Inc., Tokyo (JP)

(21) Appl. No.: 11/089,508

(22) Filed: Mar. 25, 2005

(30) Foreign Application Priority Data
Sep. 24, 2004 (JP) 2004-277714

(51) Int. Cl. C12P 21/06; C12P 19/04; C12N 9/10; C12N 1/21

(52) U.S. Cl. 435/68.1; 435/193; 435/101; 435/252.9

(57) ABSTRACT

The present invention provides a transglycosylation method and a protein catalyzing a transglycosylation reaction. A reaction of transferring a galactosylβ1→3N-acetylgalactosaminyl group from a sugar donor containing a sugar chain having a galactosylβ1→3N-acetylgalactosaminyl group bound thereto at the α-position, to a sugar chain acceptor, is carried out in the presence of a protein having an endo-α-N-acetylgalactosaminidase activity and a transglycosylation activity.

### CROSS-REFERENCE TO RELATED APPLICATIONS

The present application claims the benefit of priority based on the Japanese Patent Application No. 2004-277714 filed on Sep. 24, 2004, the entire disclosure of which is incorporated herein by reference.

### BACKGROUND OF THE INVENTION

1) Field of the Invention

The present invention relates to a transglycosylation method, a glycosidase having transglycosylation activity and applications thereof.

2) Description of the Related Art

Endo-α-N-acetylgalactosaminidase (hereinafter, “Endo-α” or “Endo-α-GalNAc-ase”) is an enzyme which recognizes a core 1 structure of an O-linked sugar chain in glycoprotein, that is, a structure having a galactosylβ1→3N-acetylgalactosamine (hereinafter, “Galβ1→3GalNAc”) disaccharide bound at the α-position to serine or threonine, and which releases the disaccharide unit by hydrolysis. The O-linked sugar chain is also called a mucin-type sugar chain and is contained in a large amount in mucin with which the surfaces of animal digestive tracts and organs are covered.

It has been reported that Endo-α producing bacteria are found in a culture of, for example, Diplococcus pneumoniae and bacteria belonging to the genus Clostridium (for example, Bhavanandan, V. P. et al., Biochem. Biophys. Res. Commum., 70, 738-745 (1976)). It has also been reported that in some Endo-α studied up to now, there is a transglycosylation activity of transferring and adding a disaccharide to an entering suitable acceptor in place of a water molecule upon hydrolysis (For example, Bardales, R. M. and Bhavanandan, V. P., J. Biol. Chem., 264, 19893-19897 (198.9)). Among microorganisms known as the Endo-α-producing bacteria, there are microorganisms whose total genome sequence has been clarified.

The presence of the Endo-α-producing microorganism is known, but there is no report on the cloning of a gene for an enzyme having the Endo-α activity. That is, the amino acid sequence of the Endo-α is not revealed, and which gene is a gene encoding the Endo-α has not been specified.

### SUMMARY OF THE INVENTION

Sugar chain are very important in the living body or the like, and in study on sugar chains, production of desired sugar chains, and production of glycoprotein, enzymes capable of transferring and adding various kinds of sugar chains have been desired. Under such circumstances, the object of the present invention is to provide a novel enzyme capable of transferring and adding a sugar chain and a method of transferring a sugar chain, that is, a method of transglycosylation.

The Endo-α is generally a hydrolase, and the present inventors have found an enzyme having extremely high transglycosylation activity from the Endo-α enzymes. Further, they have succeeded in specifying its gene to complete the present invention. The present invention also provides a transglycosylation method and a protein catalyzing a transglycosylation reaction, to be described below.
1. A method for transglycosylation, comprising a reaction of transferring a galactosylβ1→3N-acetylgalactosaminyl group from a sugar donor containing a sugar chain having a galactosylβ1→3N-acetylgalactosaminyl group bound thereto at the α-position, to a sugar chain acceptor, in the presence of a protein having an endo-α-N-acetylgalactosaminidase activity and a transglycosylation activity.
2. The method for transglycosylation according to item 1, wherein the protein is derived from a microorganism belonging to the genus Bifidobacterium.
3. The method for transglycosylation according to item 1, wherein the protein is selected from the group of (A) and (B):

(A) a protein having an amino acid sequence described in SEQ ID NO:2, and

(B) a protein having an endo-α-N-acetylgalactosaminidase activity and a transglycosylation activity, and having the amino acid sequence that includes one or several amino acids mutation in the amino acid sequence described in SEQ ID NO: 2, the mutation being selected from the group consisting of substitution, deletion, insertion, addition, and inversion.
4. The method for transglycosylation according to item 1, comprising:

cultivating in a medium a transformed cell into which a polynucleotide encoding the protein having an endo-α-N-acetylgalactosaminidase activity and a transglycosylation activity was introduced, and accumulating the protein in the medium and/or the transformed cell thereby supplying the protein,

wherein the protein is selected from the group consisting of:

(A) a protein having an amino acid sequence described in SEQ ID NO:2, and

(B) a protein having an endo-α-N-acetylgalactosaminidase activity and a transglycosylation activity, and having the amino acid sequence that includes one or several amino acids mutation in the amino acid sequence described in SEQ ID NO: 2, the mutation being selected from the group consisting of substitution, deletion, insertion, addition, and inversion.
5. The method for transglycosylation according to any one of items 1 to 4, wherein the sugar chain acceptor is selected from the group consisting of a monosaccharide, an oligosaccharide, an alcohol (1-alkanol), an amino acid and a polypeptide.
6. A method of modifying a food,

wherein the food before modification contains a sugar chain acceptor selected from the group consisting of a monosaccharide, an oligosaccharide, an alcohol (1-alkanol), an amino acid and a polypeptide,

the method comprising a reaction of transferring a galactosylβ1→3N-acetylgalactosaminyl group from a sugar donor containing a sugar chain having a galactosylβ1→3N-acetylgalactosaminyl group bound thereto at the α-position, to the sugar chain acceptor, in the presence of a protein having an endo-α-N-acetylgalactosaminidase activity and a transglycosylation activity.
7. A method of modifying a drug,
   wherein the drug before modification contains a sugar chain acceptor selected from the group consisting of a monosaccharide, an oligosaccharide, an alcohol (1-alkanol), an amino acid and a polypeptide,

the method comprising a reaction of transferring a galactosylβ1→3N-acetylgalactosaminyl group from a sugar donor containing a sugar chain having a galactosylβ1→3N-acetylgalactosaminyl group bound thereto at the α-position, to the sugar chain acceptor, in the presence of a protein having an endo-α-N-acetylgalactosaminidase activity and a transglycosylation activity.
8. A protein derived from a microorganism belonging to the genus Bifidobacterium and having an endo-α-N-acetylgalactosaminidase activity and a transglycosylation activity.
9. A protein selected from the group consisting of (A) and (B):

(A) a protein having an amino acid sequence described in SEQ ID NO:2, and

(B) a protein having an endo-α-N-acetylgalactosaminidase activity and a transglycosylation activity, and having the amino acid sequence that includes one or several amino acids mutation in the amino acid sequence described in SEQ ID NO: 2, the mutation being selected from the group consisting of substitution, deletion, insertion, addition, and inversion.
10. A polynucleotide encoding a protein selected from the group consisting of (A) and (B):

(A) a protein having an amino acid sequence shown in SEQ ID NO:2, and

(B) a protein having an endo-α-N-acetylgalactosaminidase activity and a transglycosylation activity, and having the amino acid sequence that includes one or several amino acids mutation in the amino acid sequence described in SEQ ID NO: 2, the mutation being selected from the group consisting of substitution, deletion, insertion, addition, and inversion.
11. A polynucleotide selected from the group consisting of (a) and (b):

(a) a polynucleotide consisting of a nucleotide sequence shown in SEQ ID NO: 1, and

(b) a polynucleotide which hybridizes under stringent conditions with a polynucleotide consisting of a nucleotide sequence complementary to a nucleotide sequence shown in SEQ ID NO:1, and which encodes a protein having an endo-α-N-acetylgalactosaminidase activity and a transglycosylation activity.
12. A recombinant polynucleotide containing the polynucleotide according to item 10.
13. A transformed cell containing the recombinant polynucleotide according to item 12.
14. A method for producing a protein, comprising:

culturing the transformed cell-according to item 13 in a medium and accumulating a protein having an endo-α-N-acetylgalactosaminidase activity and a transglycosylation activity in the medium and/or the transformed cell.

According to the present invention, there are provided a new transglycosylation method and a protein catalyzing a transglycosylation reaction. The present invention may be utilized for industry related to the sugar chain engineering such as production for glycoprotein.

### BRIEF DESCRIPTION OF THE DRAWINGS

FIG. 1 is a schematic diagram of the transglycosylation reaction.

FIG. 2 shows the results of measurement of hydrolysis activity.

FIG. 3 shows the results of measurement of transglycosylation activity.

FIG. 4 Shows the results of measurement of transglycosylation activity into 1-alkanol.

### DETAILED DESCRIPTIONS

Hereinafter, the present invention is described in more detail with reference to exemplary embodiments. For carrying out biochemical or genetic engineering techniques in the present invention, reference is made to various experimental manuals such as Molecular Cloning: A LABORATORY MANUAL, 3rd edition, Cold Spring Harbor Laboratory Press, Cold Spring Harbor, N.Y. (2001); “Shin Idenshi Kogaku Handbook (New Genetic Engineering Handbook)”, by Masami MURAMATSU, et al, Yodosha, Experimental Medicine, extra issue, 3rd edition, 1999; “Tanpakushitsu Jikken No Susumekata (How to Advance Protein Experiment)”, by Masato OKADA and Kaori MIYAZAKI, Yodosha, 1st edition, 1998; and “Tanpakushitsu Jikken Note (Protein Experimental Note)” by Masato OKADA and Kaori MIYAZAKI, Yodosha, 2nd edition, 1999.

In the transglycosylation method of the present invention, a protein having endo-α-N-acetylgalactosaminidase activity and transglycosylation activity is used as a catalyst to transfer a sugar chain from a sugar chain donor to a sugar chain acceptor. The sugar chain is transferred from the sugar chain donor and fed to a transfer reaction system, and the sugar chain acceptor accepts the sugar chain supplied by the sugar chain donor. The sugar chain donor contains a galactosyl β1→3N-acetylgalactosaminyl α-group. Galactosyl β1→3N-acetylgalactosamine is a disaccharide having galactose and N-acetylgalactosamine bound via β (1→3). In this specification, galactosyl β1→3N-acetylgalactosamine is also referred to hereinafter as Galβ1-3GalNAc, and the galactosyl β1→3N-acetylgalactosaminyl α-group is also referred to hereinafter as Galβ1-3GalNAcα-group.

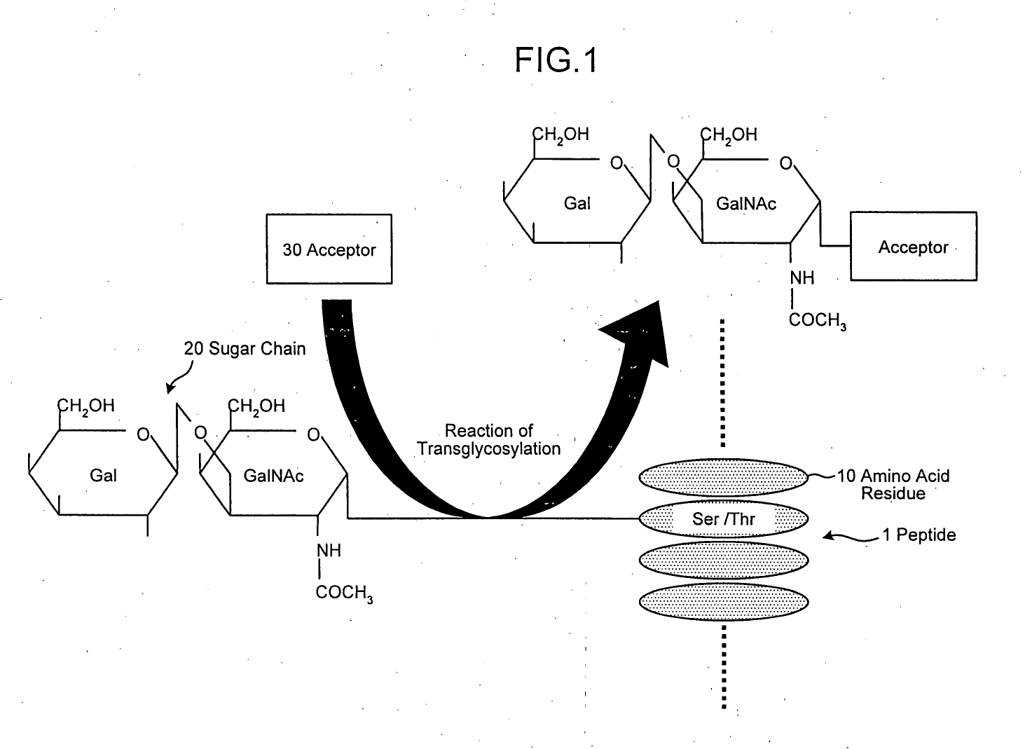

The protein used in the method for transglycosylation of the present invention has at least two activities, that is, endo-α-N-acetylgalactosaminidase activity and transglycosylation activity. Endo-α-N-acetylgalactosaminidase is an enzyme which recognizes a core 1 structure of an O-linked sugar chain in glycoprotein, that is, a structure having a β1-3GalNAc disaccharide bound at the α-position to serine or threonine, and which has at least an activity of catalyzing a reaction of releasing the disaccharide unit by hydrolysis. The O-linked sugar chain is also called a mucin-type sugar chain and is contained in a large amount in mucin with which the surfaces of animal digestive tracts and organs are covered. The protein used in the present invention also has transglycosylation activity. As shown in FIG. 1, the protein catalyzing the transglycosylation reaction in the transglycosylation method of the present invention can recognize at least a structure wherein a sugar chain moiety 20 of Galβ1-3GalNAc group in a glycoprotein (peptide 1) is bound at the α-position to serine or threonine, and which has an activity of transferring the Galβ1-3GalNAc group to an acceptor 30. The transglycosylation activity of the protein used in the present invention is high. The protein exhibiting a transglycosylation degree (%) of at least 2%, preferably 5% or more, more preferably 10% or more, still more preferably 15% or more, as determined by a method shown later in the Examples, is used.

The terms “in the presence of the protein” means that the reaction system is placed under conditions where the transglycosylation reaction can be carried out by the catalytic action of this protein. The protein may be supplied for example by adding a microorganism and/or the enzyme to the reaction system. That is, the microorganism and/or the enzyme may be allowed to be present in the reaction system in any form, provided that the microorganism and/or the enzyme effects the reaction of transferring galactosyl β1→3N-acetylgalactosaminyl group from a galactosyl β1→3N-acetylgalactosaminyl α-group-containing sugar chain donor to a sugar chain acceptor. Either of the microorganism or enzyme may be used, or both of them may be present.

The “microorganism and/or the enzyme” may be in the following form. The specific form may include a culture of the microorganisms, microbial cells separated from the culture, and a treated microbial cell product. The culture of the microorganisms is a material obtained by cultivating the microorganisms, and refers more specifically to a mixture of the microbial cells, a culture used in cultivating the microorganisms, and substances formed by the microorganism. The microbial cells may be used as a washed microorganisms after washing the cells. The treated microbial cell product may include materials obtained by disrupting, lyzing or freeze-drying the bacterium, as well as a crudely purified enzyme recovered by treating the microorganism etc. or a purified enzyme obtained by further purification. As the enzyme subjected to purification treatment, it is possible to use a enzyme such as partially purified enzyme obtained by various purification methods or an immobilized enzyme having such enzyme immobilized by a covalent bonding method, an adsorption method, an inclusion method or the like. Some of the used microorganisms are lyzed during cultivation, and in this case, a supernatant of the culture may also be utilized as a material containing the protein having transglycosylation activity.

The conditions in the reaction system using the microorganism and/or the enzyme may be suitably regulated depending on the condition such as specific type of microorganism, enzyme and starting material used. The amount of the microorganism and/or enzyme used may be an amount (effective amount) to exhibit the desired effect. This effective amount can be easily determined in a preliminary experiment which can be carried out easily by those skilled in the art; for example, when the enzyme is used, about 0.01 to 100 units (U) is preferable, and when the washed microorganisms are used, about 0.1 to 500 g/L is preferable. The reaction is carried at a temperature in the range where the enzyme used is active; that is, the reaction temperature is preferably in the range of 10 to 70° C., more preferably 20 to 65° C., still more preferably 25 to 60° C. The pH value of the enzyme reaction solution is regulated usually in the range of 2 to 12, preferably 4 to 10, more preferably 5 to 6.

The protein catalyzing the reaction described above may be obtained from a microorganism belonging to the genus Bifidobacterium. More specific example may include preferably Bifidobacterium longum, more preferably Bifidobacterium longum JCM1217. The stains given JCM numbers have been deposited with Japan Collection of Microorganisms, Riken Bioresource Center, (Hirosawa 2-1, Wako City, Saitama Prefecture, 351-0198, Japan), and may be obtained according to a predetermined procedure.

More specific examples of the protein catalyzing the transglycosylation reaction may include the following protein (A) and (B):
(A) a protein having an amino acid sequence of SEQ ID NO:2, or
(B) a protein having an endo-α-N-acetylgalactosaminidase activity and a transglycosylation activity, and having the amino acid sequence that includes one or several amino acids mutation in the amino acid sequence of SEQ ID NO: 2, the mutation being selected from the group consisting of substitution, deletion, insertion, addition, and inversion.

The present inventors newly isolated a protein having the amino acid sequence of SEQ ID NO:2 from Bifidobacterium longum JCM1217, and specified the amino acid sequence of the protein catalyzing the above reaction.

In the production method of the present invention, a protein which is substantially the same as the protein shown in (A) above may also be used. Specifically, the protein shown in (B) is provided. Herein, the term “several” refers to the number of amino acids within a range, which have less effect on the three-dimensional structure of the protein composed of amino-acid residues and the activity of the protein. It may be depending on the location and the types of amino-acid residues in the three-dimensional structure of the protein, and the range is specifically 2 to 50, preferably 2 to 30, and several preferably 2 to 10. Note that it is desirable that among those of protein groups (B), the protein having the amino acid sequence containing mutation of one or several amino acid residues selected from the group of consisting of substitution, deletion, insertion, addition, and inversion, retain approximately 50% or more, more preferably 80% or more, and further preferably 90% or more of enzyme activity of the protein containing no mutation under the conditions, 50° C. and pH8.

The mutation in amino acids as shown in the aforementioned (B) may be induced by modifying the nucleotide sequence so that the amino acids at the corresponding sites of the enzyme gene may be substituted, deleted, inserted, and/or added using, for example, the site-specific mutation method. Alternatively, the polynucleotide having modified nucleotide sequence may be obtained by the conventionally known the mutation process. The mutation process may include a technique involving a step for in vitro inducing the mutation in the DNA encoding the amino acids of the (A) under the treatment of hydroxylamine and a technique involving a step for introducing the mutation in a bacterium belonging to the genus Escherichia containing the DNA encoding the amino acids of the (A) by UV irradiation, or under the treatment with N-methyl-N′-nitro-N-nitrosoguanidine(NTG) or any of mutating agents generally used for mutation engineering, such as nitrous acid.

Mutations such as substitution, deletion, insertion, addition and inversion in amino acids, accompanying the modification to the nucleotide sequence, may include naturally occurring mutations such as differences due to microbial species or microbial strain. By expressing such mutated DNA in a suitable cell and examining the activity of the present enzyme in the expression product, a DNA encoding substantially the same protein as the protein of SEQ ID NO:2 is obtained.

The polynucleotide encoding the protein used in the transglycosylation method of the present invention includes a polynucleotide encoding the amino acid sequence shown in SEQ ID NO:2. By codon degeneracy, there can be a plurality of nucleotide sequences encoding one amino acid sequence. That is, the polynucleotide of the present invention includes polynucleotides having nucleotide sequences encoding the following proteins:
(A) a protein having an amino acid sequence of SEQ ID NO:2, and
(B) a protein having an endo-α-N-acetylgalactosaminidase activity and a transglycosylation activity, and having the amino acid sequence that includes one or several amino acids mutation in the amino acid sequence of SEQ ID NO: 2, the mutation being selected from the group consisting of substitution, deletion, insertion, addition, and inversion.

The nucleotide sequence encoding the amino acid sequence of SEQ ID NO:2 is exemplified by the nucleotide sequence of SEQ ID. NO:1. Further, as the polynucleotide which is substantially the same as the DNA having the nucleotide sequence of SEQ ID NO: 1, there may be mentioned a polynucleotide substantially the same as the polynucleotide having the nucleotide sequence shown in SEQ ID NO: 1 and obtained by isolating a polynucleotide which hybridizes under stringent conditions with a polynucleotide consisting of, or with a probe prepared from, a nucleotide sequence complementary to the nucleotide sequence shown in SEQ ID NO: 1 and which encodes a protein having an endo-α-N-acetylgalactosaminidase activity and a transglycosylation activity, from a polynucleotide encoding the protein having the amino acid sequence shown in SEQ ID NO:2 or from a cell retaining the polynucleotide.

Specific examples of the polynucleotide of the present invention preferably include the following polynucleotides (a) and (b):
(a) a polynucleotide having a nucleotide sequence shown in SEQ ID NO:1 in the Sequence Listing, and
(b) a polynucleotide which hybridizes under stringent conditions with a polynucleotide consisting of a nucleotide sequence complementary to the nucleotide sequence of SEQ ID NO:1, and which encodes a protein having an endo-α-N-acetylgalactosaminidase activity and a transglycosylation activity.

The probe may be prepared in an ordinary method-for example on the basis of the nucleotide sequence shown in SEQ ID NO:1. The method of probing, which the desired polynucleotide is isolated by screening a polynucleotide hybridizing with the probe, may also be carried out in an ordinary method. For example, the DNA probe may be prepared by amplifying the nucleotide sequence cloned in a plasmid or a phage vector, cleaving with a restriction enzyme a nucleotide sequence intended to be used as the probe, and extracting it. The site to be cleaved off maybe regulated depending on the desired DNA.

As used herein, the “stringent conditions” refers to those conditions under which a specific hybrid is formed whereas an unspecific hybrid is not formed. These conditions are hardly expressed explicitly numerically, but by way of example, there may be mentioned those conditions under which DNA molecules having higher homology e.g. preferably 50% or more, more preferably 80% or more, still more preferably 90% or more, further more preferably 95% or more homology, hybridize with each other while DNA molecules having lower homology do not hybridize with each other, or those conditions under which hybridization occurs under usual washing conditions in Southern hybridization, that is, at a salt concentration corresponding to 1×SSC and 0.1% SDS at 60° C., preferably 0.1×SSC and 0.1% SDS at 60° C. Among the genes hybridizing under such conditions, there are genes having a stop codon in their sequence or encoding enzymes whose activity is lost due to a mutation in active center, and such genes can be easily removed by ligating each of them into a commercial expression vector, expressing it in a suitable host, and measuring the enzyme activity of each expression product by a method described later in the Examples.

As described above, a polynucleotide having the nucleotide sequence encoding the protein (B), and the polynucleotide (b), desirably keep about at least half of the enzyme activity of a protein having the amino acid sequence of SEQ ID. NO:1, more preferably 80% or more, still more preferably 90% or more of the transglycosylation activity.

The DNA having the nucleotide sequence in SEQ ID NO:1 may be obtained from chromosomal DNA of Bifidobacterium longum JCM1217 or from a DNA library by PCR (polymerase chain reaction; see White, T. J. et al. in Trends Genet. 5, 185 (1989)) or by hybridization. The primers used in PCR may be designed based on an internal amino acid sequence determined on the basis of, for example, the purified protein having a peptide synthesizing activity. As PCR primers, those having sequences corresponding to the 5′-non-translating region and 3′-non-translating region respectively may be used to amplify a full-length coding region of the present protein.

The primers may be synthesized in a usual manner according to, for example, the phosphoamidite method (see Tetrahedron Letters (1981), 22, 1859) in a DNA synthesizer model 380B manufactured by Applied Biosystems. The PCR reaction may be carried out using, for example, Gene Amp PCR system 9600 (supplied from PERKIN ELMER) and TaKaRa La PCR in vitro Cloning Kit (supplied from TAKARA BIO INC.) by a method prescribed by a supplier such as each manufacturer.

Furthermore, for homologues of the protein having transglycosylation activity derived from Bifidobacterium longum JCM1217, nucleotide sequences for the protein in Example (8-4) below were specified by the present inventors.

The sugar chain donor used in the transglycosylation method of the present invention may be a compound having a Galβ1-3GalNAc group as a sugar chain, and examples may include mucin, a protein having a Galβ1-3GalNAc group as a sugar chain, more specifically a protein having a structure where a disaccharide Galβ1-3GalNAc is bound at the α-position to serine or threonine, as well as Galβ1-3GalNAc group-containing p-nitrophenol (referred to hereinafter as Galβ1-3GalNAc α1-pNP; p-Nitrophenol is referred to as pNP).

The sugar chain acceptor used in the transglycosylation method of the present invention includes, for example, monosaccharides, oligosaccharides, alcohols (1-alkanol), amino acids and polypeptides, preferably saccharides such as monosaccharides and oligosaccharides, more preferably glucose, galactose, mannose, and arabinose.

In one preferable embodiment of the transglycosylation method of the present invention, a transformant expressing the protein is prepared and used in the transglycosylation method. That is, transformed cells into which a polynucleotide encoding the protein (A) or (B) described above, or the polynucleotide (a) or (b) described above, was introduced-are cultivated in a medium to accumulate a protein having an endo-α-N-acetylgalactosaminidase activity and a transglycosylation activity in the medium and/or the transformed cells thereby supplying the protein to the reaction system for transglycosylation.

As the host for expressing the protein specified by the polynucleotide having the nucleotide of SEQ ID NO: 1, it is possible to use various procaryotic cells including bacteria of the genus Escherichia, such as Escherichia coli, those of the genus Empedobacter, those of the genus Sphingobacterium, those of the genus Flavobacterium, and Bacillus subtilis, as well as various eucaryotic cells including Saccharomyces cerevisiae, Pichia stipitis, and Aspergillus oryzae.

A recombinant DNA used in introducing the DNA having the nucleotide sequence in SEQ ID NO:1 into a host can be prepared by inserting the DNA into a vector compatible with the host in such a form that the protein encoded by the DNA can be expressed. When a promoter inherent in the gene encoding the above protein derived from Bifidobacterium longum acts in the intended host cell, the promoter may be used for expressing the protein. If necessary, another promoter-working in the host cell may be ligated to the DNA of SEQ ID NO:1 and expressed under its control.

The transformation method of introducing a recombinant such as an expression vector into a host cell may include the D. M. Morrison method (Methods in Enzymology 68, 326 (1979)), a method that involves treating a recipient microbial cell with calcium chloride to increase the permeability of DNA (Mandel, M. and Higa, A., J. Mol. Biol., 53, 159 (1970)).

The transglycosylation method of the present invention is used preferably in modifying foods, pharmaceutical preparations and various physiologically active substances. In a preferable embodiment of the method of modifying a food or pharmaceutical preparation, a sugar chain is transferred and added to a part or the whole of a component incorporated into the food or pharmaceutical preparation, to produce a modified food or pharmaceutical preparation containing the component having the sugar chain added thereto. The component to which the sugar chain is to be added is a sugar chain acceptor. Preferable examples of the sugar chain acceptor and donor include those described above.

### EXAMPLES

Hereinafter, the present invention is described in more detail with reference to the following Examples, but the present invention is not limited thereto.

(1) Analysis of Sugar Chain by TLC (Thin Layer Chromatography)

TLC was performed by using Silica gel 60 (MERCK) as the plate with a mixture of developing solvents of chloroform/methanol/water (3/3/1) and a coloration reagent with the composition shown in Table 1.

TABLE 1

| | | |
|---|---|---|
| Acetone | 10 | ml |
| Aniline | 100 | μl |
| Diphenylamine | 0.1 | g |
| Phosphoric acid | 1 | ml |

(2) Analysis of a Reaction Product by HPLC (High Performance Liquid Chromatography)

A reaction mixture was subjected to HPLC (supplied from Hitachi D-7420 UV-VIS Detector, D-7100 Pump, D-7500 Integrator) to analyze the reaction product. Analysis was performed under the following conditions according to a manual attached to the apparatus. A normal-phase column was used with Solvent A/Solvent B (25/75) as the solvent.
Column; TSK-gel amide-80
Solvent A; 100% H₂O
Solvent B; 100% CH₃CN
Column temp; 40° C.
Flow rate; 1 mL/min
Wavelength; 214 nm
   (3) Determination of Enzyme Activity
   (3-1) Determination of Hydrolysis Activity

The enzymatic reaction was performed using Galβ1-3GalNAcα1-pNP was used as the substrate, and liberated pNP (p-nitrophenol) was quantified calorimetrically under alkali conditions. Specifically, a sample was reacted at 37° C. for a predetermined time, and Na₂CO₃ was added to terminate the reaction, followed by measurement of the absorbance at 400 nm. The composition of the reaction solution is shown below (Table 2). When another substrate was used to examine substrate specificity, it was used at the same concentration as that of Galβ1-3GalNAcα1-pNP below.

TABLE 2

| | | |
|---|---|---|
| 10 mM Galβ1-3GalNAcα1-pNP | 0.5 | μl |
| 1 M acetate buffer (pH 5.0) | 1.0 | μl |
| Water | 16 | μl |
| Enzyme | 2.5 | μl |
| + | | |
| Na₂CO₃ | 30 | μl |

One unit was defined as the amount of the enzyme necessary to release 1 μmol pNP at 37° C. per minute, and the activity was acquired according to the following equation (I):
Units/ml=A/(18.3 TV) (I),
wherein
A=absorbance (nm)
T=reaction time (min)
V=amount of the enzyme in the total volume including the termination solution
18.3=millimolar absorptivity coefficient of pNP at pH 10 or more

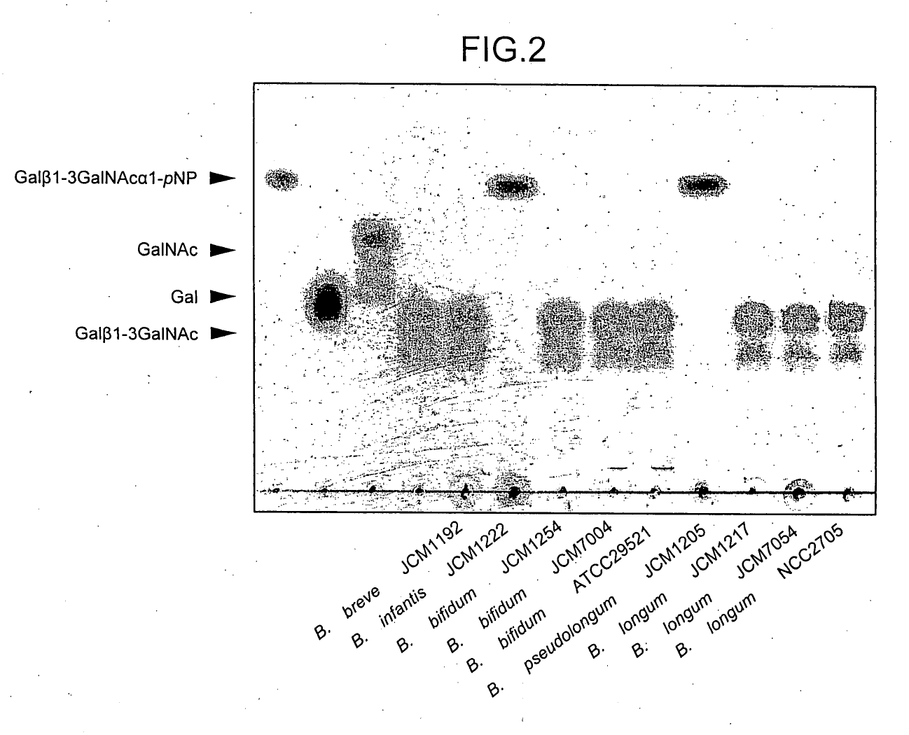

Various bifidobacteria were examined by using galactosylβ1-3N-acetylgalactosaminyl α1-p-nitrophenol (Galβ1-3GalNAc α1-pNP) as the substrate, to determine whether they had the activity of the present enzyme or not. The bifidobacteria were cultivated by stationary culture in GAM bouillon (supplied from Nissui Pharmaceutical) at 37° C. for 24 hours under anaerobic conditions. When the reaction solution was subjected to TLC, the activity of the present enzyme in decomposing the substrate not into monosaccharide units but into disaccharide units was recognized in many strains, as shown in FIG. 2. In FIG. 2, the 4th lane (Galβ1-3GalNAc) from the left is a control of disaccharide obtained by cutting the same substrate with an enzyme derived from a bacterium of the genus Bacillus known as an endo-α-N-acetylgalactosaminidase-producing bacterium.

With respect to this reaction product and GalNAc, it appears that there are two separated spots, but this is estimated due to α-difference in anomer. The present inventors separately confirmed that one spot appeared when a developing solvent and coloration reagent different from those described above were used.

(3-2) Determination of Transglycosylation Activity

The present enzyme derived from Bifidobacterium longum JCM1217 was examined for its transglycosylation activity in the following manner. In the reaction system for determining the transglycosylation activity, the pNP substrate was used as the sugar chain donor in every case. The reaction was performed using the sugar chain donor (final concentration, 2 mM), the acceptor (final concentration, 1 M) and the enzyme (final concentration, 61 units/L) in a 10 mM acetate buffer, pH 5.0. After the reaction was conducted at 37° C. for 20 minutes, the reaction mixture was boiled for 3 minutes to inactivate the enzyme, then passed through a filter at 4° C. and subjected to HPLC. The degree of transglycosylation was acquired by the following equation (II):
Degree of transglycosylation=Area of transglycosylation product peak/(Area of hydrolysis product peak+Area of transglycosylation product peak) (II)

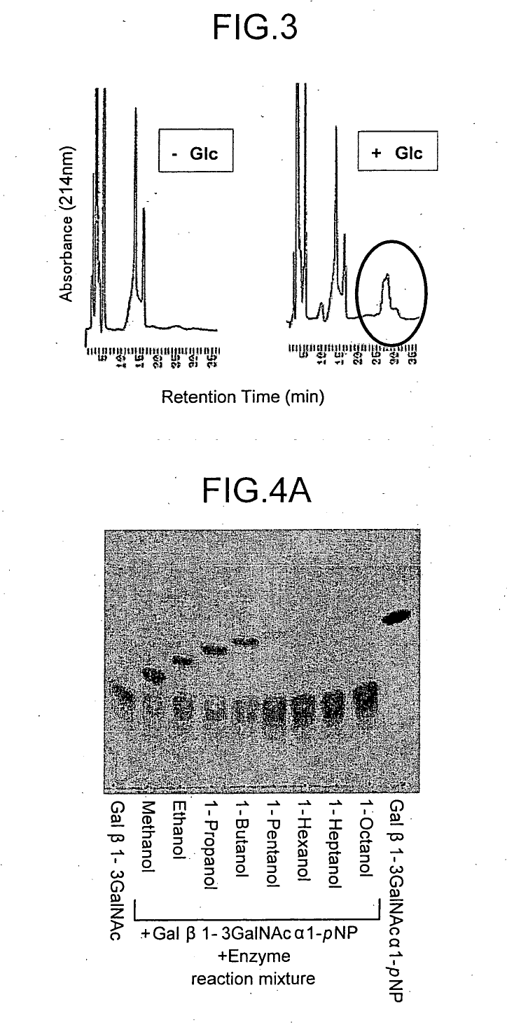

The reaction was carried out by using glucose as the acceptor and Galβ1-3GalNAcα1-pNP as the sugar chain donor, and the reaction solution was subjected to HPLC. The results are shown in FIG. 3. The left chromatograph (−Glc) is the glucose-free control, and the right chromatograph (+Glc) is the reaction solution. In the right chromatograph (+Glc), a new peak shown as the enclosed region was detected, and thus this region was isolated and analyzed by MALDI-TOF-MS (matrix assisted laser desorption ionization (MALDI); time of flight (TOF); mass spectrometry (MS)).

As a result, a peak indicating a trisaccharide having Glc added to Gal-GalNAc released from the sugar chain donor was obtained, thus revealing that the isolated enzyme had transglycosylation activity. The same HPLC analysis revealed that the sugar chain was also transferred to galactose, mannose and arabinose. The degree of transglycosylation to each acceptor under the predetermined reaction conditions is also shown (Table 3). The degree of transglycosylation to galactose, particularly to mannose, was lower than to glucose. It is estimated that a hydroxyl group at the 2- or 4-position is involved in the transglycosylation reaction.

TABLE 3

| | Degree of |
|---|---|
| Sugar chain acceptor | transglycosylation (%) |
| Glucose | 19.7 |
| Galactose | 8.3 |
| Mannose | 2.5 |
| Arabinose | 18.0 |
| Maltose | 14.4 |

(4) Method of Cultivating E. coli

As the medium used, a liquid medium LB broth (supplied from DIFCO) was used, and when an agar medium was used, the medium was prepared by adding agar at a final concentration of 2% to the above liquid medium. When ampicilin was used, it was added to a final concentration of 150 μm/ml.

(5) Construction of Expression Plasmid

On the basis of the nucleotide sequence of ORF encoding protein BL0464 of unknown functions, which was estimated as Endo-α-GalNAc-ase in Bifidobacterium longum NCC2705, primers capable of amplifying it by PCR were designed (Table 4). A restriction enzyme site was integrated in the primer to facilitate cloning. Using Bifidobacterium longum JCM1217 genome as a template, PCR was conducted using TaKaRa Ex Taq™ DNA Polymerase (supplied from TAKARA BIO INC.) in a reaction solution having the following composition and under the following conditions.

Composition of the reaction solution: template DNA, 0.01 to 0.1 μg; 2.5 units TaKaRa Ex Taq™ DNA Polymerase; 10 μL 10× Ex Taq™ buffer (supplied from TAKARA BIO INC.); 0.2 mM dNTP mixture (supplied from TAKARA BIO INC.); and 1 μM primers.

Reaction conditions: 94° C., 30 seconds→(94° C., 30 seconds→60° C., 30 seconds→72° C., 6 minutes)×30 cycles→72° C., 6 minutes.

TABLE 4

| | |
|---|---|
| BL0464 forward primer | 5′-TTAACCTCCATGGGCAGCGGGGGAGG-3′ |
| (NcoI-endo-α-GalNAc-ase) | :SEQ ID NO; 13 |
| BL0464 reverse primer | 5′-AACCTGCGGCCGCCAGTTGCTCGCGATTGC-3′ |
| (Endo-α-GalNAc-ase-His₆-tag-NotI) | SEQ ID NO; 14 |

The resulting PCR amplification product (about 6 kbp) was subjected to TA cloning, and then cleaved with restriction enzymes Nco I (supplied from Toyobo) and Not I (supplied from Toyobo) simultaneously, and the resulting fragment (about 6 kb) was inserted between Nco I/Not I sites in E. coli expression vector pET-23d(+) (supplied from Novagen). Ligation was carried out using Ligation High (supplied from TAKARA BIO INC.).

(6) Transformation of E. coli

Transformation of E. coli was conducted by adding the ligation reaction solution (about 20 μL) to competent cells (100 to 200 μL) then giving heat shock to the cells at 42° C. for 45 seconds, adding 800 to 900 μL SOC medium to the cells and cultivating the cells at 37° C. for 1 hour. The resultant culture was suitably diluted and applied onto an antibiotic-containing LB agar plate, and whether transformation occurred or not was confirmed by subjecting the growing E. coli to colony PCR, or by plasmid extraction and subsequent treatment with restriction enzymes. In extraction of the plasmid, Wizard plus SV Minipreps (supplied from Promega) was used. The E. coli competent cells were prepared by the Inoue method and stored at −80° C.

(7) Expression and Purification of Recombinant Endo-α-GalNAc-ase

E. coli BL21 (DE3) /pET-23d-Endo-α-GalNAc-ase was subjected to shake culture in LB-ampicilin medium at 37° C. until the OD600 of the culture reached 0.4, and then IPTG was added at a final concentration of 0.4 mM, and shake culture was continued for 3 hours, whereby the desired Endo-α-GalNAc-ase was expressed in a large amount. The bacterial cells were collected and used to prepare a cell-free extract fraction with Bug Buster reagent (supplied from Novagen), and its activity was confirmed.

For purification, the cell-free extract fraction was added to a nickel column HiTrap™ Chelating HP (supplied from Amersham) previously equilibrated with a buffer (2 mM Tris-HCl buffer (pH 8.0), 0.5 M NaCl) containing 5 mM imidazole, and an excess of protein in the column was washed away with the same buffer containing 5 mM imidazole, and subsequently the column was connected to FPLC. In FPLC, a buffer containing 20 mM imidazole, in an amount of 10 cv (column volume), was passed through the column and then the desired Endo-α-GalNAc-ase was eluted with a buffer containing 5cv 1 M imidazole. The eluted fractions were dialyzed against a 10 mM KPB buffer, pH 7.0, and the desired Endo-α-GalNAc-ase was identified by activity measurement and SDS-PAGE.

(8) Analysis of DNA Sequence

Gene manipulation was carried out mainly in accordance with the method of Sambrook et al. The analysis of DNA sequence was carried out according to the method of Sanger et al. by using BigDye terminator v3.0 cycle sequencing ready reaction kit and ABI prism™ 310 NT genetic analyzer (Applied Biosystems).

The full-length nucleotide sequence of ORF encoding the Endo-α-GalNAc-ase in the genome of Bifidobacterium longum JCM1217 was determined.

(8-1) Sequence of the Gene Fragment Integrated in the Vector

For sequencing of the whole region of the desired gene fragment integrated in the vector, a primer binding region in the vector was utilized, and using T7 promoter primer and M13 reverse primer, sequences at both ends of the gene fragment were decoded. On the basis of the revealed sequences, primers directing towards the inside of the gene fragment were designed to advance decoding. By repeating this procedure, the nucleotide sequence of the whole region of the gene fragment integrated in the vector was determined. Given the gene fragment integrated in the vector, PCR amplification was initiated from the primer sequence, and thus the N-terminal region and C-terminal region of full-length ORF encoding Endo-α-GalNAc-ase derived from Bifidobacterium longum JCM1217 could not be completely determined.

(8-2) Sequence of C-Terminal Region

To utilize homology among Bifidobacterium longum species, a primer (BL0464-C152 reverse primer: 5′-TGCGATTCATCGCCTAGCAG-3′ (SEQ ID NO:15)) corresponding to the slightly outside of the C-terminus to the inside of ORF was designed on the basis of information on a nucleotide sequence of Bifidobacterium longum NCC2705. This primer, and the primer in Endo-α-GalNAc-ase gene fragment integrated in the vector, were used for PCR with Bifidobacterium longum JCM1217 genome as the template thereby to amplify the gene fragment containing the C-terminal region, which was then subjected to TA cloning using T easy vector. By sequencing, the nucleotide sequence of the complete C-terminal region in ORF encoding Endo-α-GalNAc-ase derived from Bifidobacterium longum JCM1217 was determined.

(8-3) Sequence of N-Terminal Region

Because the outside of the N-terminus was composed of a repeat region, a suitable primer could not be designed, so that by using Southern blotting, a group of N-terminal region-containing gene fragments was obtained by restriction enzyme treatment from the genome of Bifidobacterium longum JCM1217. A vector having such fragment ligated therein was transformed into a competent cell. From the growing E. coli, positive clones were selected by colony PCR. By sequencing, the nucleotide sequence of the complete N-terminal region in ORF encoding Endo-α-GalNAc-ase derived from Bifidobacterium longum JCM1217 was determined.

(8-4)

On the basis of the information on the nucleotide sequence or amino acid sequence of the protein specified in Bifidobacterium longum JCM1217, homologue sequences were specified in the following microbial strains. Each microbial strain and the sequence number of its nucleotide sequence are shown. Each sequence can be searched from a database such as DDBJ (DNA Data Bank of Japan) by using each accession number etc.
Streptococcus pneumoniae R6 (Accession No. AE008413); SEQ ID NO:3
Bifidobacterium longum NCC2705 (Accession No. AE014666); SEQ ID NO:5
Clostridium perfringens str.13 (Accession No. AP003187); SEQ ID NO:7
Streptomyces coelicolor A3 (2) (Accession No. AL939127); SEQ ID NO:9
Enterococcus faecalis V583 (Accession No. AE016952); SEQ ID NO:11
   (9) Transglycosylation into 1-alkanol

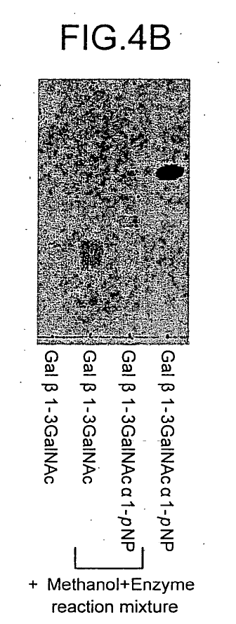

The transglycosylation experiment described in Example (3-2) was performed with various kinds of 1-alkanol as the acceptor. The reaction system for measurement of transglycosylation activity was carried out by using the sugar chain donor Galβ1-3GalNAcα1-pNP at a final concentration of 2 mM, the acceptor 1-alkanol at a final concentration of 15% (v/v) and the purified enzyme at a final concentration of 6.8 units/L in a 50 mM potassium phosphate buffer, pH 7.0. After the reaction at 37° C. for 60 minutes, 3 μl of the reaction solution was spotted on a TLC plate, developed with a mixture of solvent of chloroform/methanol/water (7/6.2/2), and colored with naphthoresorcinol in sulfuric acid (0.2% (w/v) naphthoresorcinol in sulfuric acid:ethanol (5:95 (v/v)). The results are shown in FIG. 4A. As shown in the drawing, when 1-alkanol was added as the acceptor, new spots were detected in addition to Galβ1-3GalNAc as a hydrolysis product of the sugar chain donor, and these spots were detected at different mobility depending on the type of 1-alkanol. Therefore it was considered that the spots was due to the substance of Galβ1-3GalNAc transferred to each alkanol.

Using methanol as the sugar chain acceptor, the reaction with Galβ1-3GalNAc and the reaction with Galβ1-3GalNAcα1-pNP were then carried out respectively in order to confirm whether the formation of these substances by the enzyme reaction was due to the transglycosylation reaction or due to the reverse reaction of hydrolysis reaction. The reaction conditions were the same as described above except that Galβ1-3GalNAc and Galβ1-3GalNAcα1-pNP were used at a final concentration of 1.2 mM respectively. The results are shown in FIG. 4B. As shown in the drawing, the spot formed in FIG. 4A was not formed in the reaction of Galβ1-3GalNAc with methanol, but formed in the reaction of Galβ1-3GalNAcα1-pNP with methanol, thus revealing-that the products were formed not by the reverse reaction of hydrolysis reaction but by the transglycosylation reaction.

Although the present invention has been described with reference to the preferred examples, it should be understood that various modifications and variations can be easily made by those skilled in the art without departing from the spirit of the invention. Accordingly, the foregoing disclosure should be interpreted as illustrative only and is not to be interpreted in a limiting sense. The present invention is limited only by the scope of the following claims along with their full scope of equivalents.

1. A method for transglycosylation, comprising a reaction of transferring a galactosylβ1→3N-acetylgalactosaminyl group from a sugar donor containing a sugar chain having a galactosylβ1→3N-acetylgalactosaminyl group bound thereto at the α-position, to a sugar chain acceptor, in the presence of a protein having an endo-α-N-acetylgalactosaminidase activity and a transglycosylation activity.

2. The method for transglycosylation according to claim 1, wherein the protein is derived from a microorganism belonging to the genus Bifidobacterium.

3. The method for transglycosylation according to claim 1, wherein the protein is selected from the group of (A) and (B):
(A) a protein having an amino acid sequence described in SEQ ID NO:2, and
(B) a protein having an endo-α-N-acetylgalactosaminidase activity and a transglycosylation activity, and having the amino acid sequence that includes one or several amino acids mutation in the amino acid sequence described in SEQ ID NO: 2, the mutation being selected from the group consisting of substitution, deletion, insertion, addition, and inversion.

4. The method for transglycosylation according to claim 1, comprising:
cultivating in a medium a transformed cell into which a polynucleotide encoding the protein having an endo-α-N-acetylgalactosaminidase activity and a transglycosylation activity was introduced, and accumulating the protein in the medium and/or the transformed cell thereby supplying the protein,
wherein the protein is selected from the group consisting of:
(A) a protein having an amino acid sequence described in SEQ ID NO:2, and
(B) a protein having an endo-α-N-acetylgalactosaminidase activity and a transglycosylation activity, and having the amino acid sequence that includes one or several amino acids mutation in the amino acid sequence described in SEQ ID NO: 2, the mutation being selected from the group consisting of substitution, deletion, insertion, addition, and inversion.

5. The method for transglycosylation according to claim 1, wherein the sugar chain acceptor is selected from the group consisting of a monosaccharide, an oligosaccharide, an alcohol (1-alkanol), an amino acid and a polypeptide.

6. A method of modifying a food,
wherein the food before modification contains a sugar chain acceptor selected from the group consisting of a monosaccharide, an oligosaccharide, an alcohol (1-alkanol), an amino acid and a polypeptide,
the method comprising a reaction of transferring a galactosyl β1→3N-acetylgalactosaminyl group from a sugar donor containing a sugar chain having a galactosylβ1→3N-acetylgalactosaminyl group bound thereto at the α-position, to the sugar chain acceptor, in the presence of a protein having an endo-α-N-acetylgalactosaminidase activity and a transglycosylation activity.

7. A method of modifying a drug,
wherein the drug before modification contains a sugar chain acceptor selected from the group consisting of a monosaccharide, an oligosaccharide, an alcohol (1-alkanol), an amino acid and a polypeptide,
the method comprising a reaction of transferring a galactosylβ1→3N-acetylgalactosaminyl group from a sugar donor containing a sugar chain having a galactosylβ1→3N-acetylgalactosaminyl group bound thereto at the α-position, to the sugar chain acceptor, in the presence of a protein having an endo-α-N-acetylgalactosaminidase activity and a transglycosylation activity.

8. A protein derived from a microorganism belonging to the genus Bifidobacterium and having an endo-α-N-acetylgalactosaminidase activity and a transglycosylation activity.

9. A protein selected from the group consisting of (A) and (B):
(A) a protein having an amino acid sequence described in SEQ ID NO:2, and
(B) a protein having an endo-α-N-acetylgalactosaminidase activity and a transglycosylation activity, and having the amino acid sequence that includes one or several amino acids mutation in the amino acid sequence described in SEQ ID NO: 2, the mutation being selected from the group consisting of substitution, deletion, insertion, addition, and inversion.

10. A polynucleotide encoding a protein selected from the group consisting of (A) and (B):
(A) a protein having an amino acid sequence shown in SEQ ID NO:2, and
(B) a protein having an endo-α-N-acetylgalactosaminidase activity and a transglycosylation activity, and having the amino acid sequence that includes one or several amino acids mutation in the amino acid sequence described in SEQ ID NO: 2, the mutation being selected from the group consisting of substitution, deletion, insertion, addition, and inversion.

11. A polynucleotide selected from the group consisting of (a) and (b):
(a) a polynucleotide consisting of a nucleotide sequence shown in SEQ ID NO:1, and
(b) a polynucleotide which hybridizes under stringent conditions with a polynucleotide consisting of a nucleotide sequence complementary to a nucleotide sequence shown in SEQ ID NO: 1, and which encodes a protein having an endo-α-N-acetylgalactosaminidase activity and a transglycosylation activity.

12. A recombinant polynucleotide containing the polynucleotide according to claim 10.

13. A transformed cell containing the recombinant polynucleotide according to claim 12.

14. A method for producing a protein, comprising:
culturing the transformed cell according to claim 13 in a medium and accumulating a protein having an endo-α-N-acetylgalactosaminidase activity and a transglycosylation activity in the medium and/or the transformed cell.
